(19) United States
(12) Patent Application Publication
(10) Pub. No.: US 2023/0402180 A1
(43) Pub. Date: Dec. 14, 2023

(54) TECHNIQUES FOR GENERATING PREDICTIVE OUTCOMES RELATING TO SPINAL MUSCULAR ATROPHY USING ARTIFICIAL INTELLIGENCE

(71) Applicants: Silvia Elena MOLERO LEON, La Aurora, Heredia (CR); Hélène Jeanne SAHRI, Basel (CH); Turap TASOGLU, Arlesheim, Basel-Landschaft (CH); Cigdem TOURKMEN, Riehen, Basel City (CH)

(72) Inventors: Silvia Elena MOLERO LEON, La Aurora, Heredia (CR); Hélène Jeanne SAHRI, Basel (CH); Turap TASOGLU, Arlesheim, Basel-Landschaft (CH); Cigdem TOURKMEN, Riehen, Basel City (CH)

(21) Appl. No.: 18/035,260

(22) PCT Filed: Nov. 22, 2021
(86) PCT No.: PCT/US2021/060281

§ 371 Date: May 3, 2023

(30) Foreign Application Priority Data
Nov. 26, 2020 (EP) 20211555.6

(51) Int. Cl. G16H 50/20; G06N 3/0455; G06N 3/08; G16H 40/67

(52) U.S. Cl. CPC G16H 50/20; G16H 40/67; G06N 3/08; G06N 3/0455

(57) ABSTRACT

Disclosed are techniques for using artificial intelligence (AI) to facilitate the treatment of subjects diagnosed with spinal muscular atrophy (SMA). Methods and systems disclosed herein relate to techniques for using AI to predict the disease progression in subjects diagnosed with SMA, detect latent commonalities across subjects with SMA to identify candidate subjects for new or existing clinical studies, and intelligently select subject-specific therapeutic treatments for treating SMA.

### CROSS-REFERENCE TO RELATED APPLICATIONS

This application claims the benefit of and the priority to European Application Number 20211555.6, titled “Techniques for Generating Predictive Outcomes Relating to Spinal Muscular Atrophy using Artificial Intelligence, filed on Nov. 24, 2020, which is hereby incorporated by reference in its entirety for all purposes.

### FIELD

Methods and systems disclosed herein generally relate to techniques for using artificial intelligence (AI) to facilitate the treatment of subjects diagnosed with spinal muscular atrophy (SMA). More specifically, methods and systems disclosed herein relate to techniques for using AI to predict the disease progression in subjects diagnosed with SMA, detect hidden commonalities across subjects with SMA to identify candidate subjects for new or existing clinical studies, and intelligently select subject-specific therapeutic treatments for treating SMA.

### BACKGROUND

The brain contains specialized cells, called motor neurons, which control voluntary movement in over 500 muscles across the body. Motor neurons include axons, which are long fibers that carry signals from the brain along the spinal cord to target muscles. The health of motor neurons, however, largely depends on the existence of a protein called the survival motor neuron (SMN) protein. SMN1, a gene located on chromosome 5, produces a sufficient amount of the SMN protein to maintain healthy motor neurons.

A person with a neuromuscular disease called spinal muscular atrophy (SMA) produces an insufficient amount of the SMN protein due to a mutation in the SMN1 gene. The deficiency in the SMN protein causes the motor neurons to progressively degenerate. Degenerated motor neurons, however, prevent the brain signals for controlling voluntary movement from reaching the target muscles. While SMN1 may not produce a sufficient amount of the SMN protein, most people do have at least one functional copy of SMN1, called the SMN2 gene. SMN2 can produce about 10-20% of the normal level of the SMN protein, allowing for at least some motor neurons to survive. Those with SMA generally experience progressive muscle atrophy, mostly of proximal muscles, causing muscle weakness and decay.

SMA presents a variety of unique challenges. For instance, there is a wide diversity of symptoms and symptom severity across subjects with SMA. Defining treatment workflows for treating subjects is, therefore, particularly challenging with SMA-diagnosed subjects. SMA-related treatments can be highly contextual to the disease progression experienced by the subject, and thus, defining treatment workflows with specific treatment schedules is a challenging and complex task.

Often, defining a schedule for treating subjects is responsive to symptoms rather than predictive. For example, over the course of the disease, there is a large variability across subjects regarding which muscle groups weaken initially and to what degree. Subjects generally experience weakness in muscle groups that support the spine, imposing a burden on the respiratory system. For some subjects, however, the progression of atrophy for this muscle group is quick, whereas, for other subjects, the progression is gradual. Additionally, certain subjects experience weakness in the muscle group that supports swallowing, imposing a burden on everyday eating activity. For some subjects, the muscle group supporting swallowing weakens before the muscle group that supports the spine, whereas, for other subjects, the sequence of muscle group degeneration is reversed. Treating subjects with weakening muscles that support swallowing is very different from treating subjects with weakening muscles that support the spine. Typically defining treatment for an individual subject involves closely monitoring of the subject's symptoms and responding with treatment accordingly.

In another example illustrating challenges unique to SMA, one treatment involves increasing the expression of the SMN protein using genetic replacement therapies. Increasing the SMN protein expression, however, causes an improvement in a subject's motor function only when performed within a therapeutic window. For instance, in animal models, performing SMN restoration therapies is effective at improving motor function only if the therapy is delivered within the first three days after birth. The same therapies may not be effective at all if performed 10 or more days after birth. There is a narrow window of time to deliver certain SMN therapies for improving motor function and that window is contextual to each subject. For a new subject (e.g., patient), identifying a therapeutic window for SMN protein expression is a technically challenging and complex task. Often, identifying a treatment and treatment schedule for a new subject involves manually comparing the many different and complicated attributes of the new subject with the same of previously-treated subjects.

The severity of symptoms across subjects with SMA is also highly variable. Symptom severity can be based on various factors, including, for example, time between symptom onset and diagnosis or treatment, type of SMA, the subject's daily activities, and the like. Gaining insights into a given subject's potential severity of diagnosed SMA Type and/or the timing of future SMA-related events is difficult. For this reason, treatments may be performed far too late. Studies have found that, on average, SMA Type-I patients are diagnosed and then treated over 4 months after symptom onset, and SMA Type-III patients are diagnosed and then treated over 10 months after symptom onset.

In addition, the lack of availability of data is another unique challenge in the SMA context. SMA is characterized as a rare disease because the disease affects approximately one in 10,000 births. An experienced physician may never have the opportunity to treat a subject with SMA over his or her entire career. Even at a regional level, the number of previously-treated subjects with SMA may be limited. A physician treating a subject who is newly diagnosed with SMA may not have access to a sufficient amount of data to inform a new treatment schedule for the new subject. Further, testing new treatments on SMA subjects using clinical studies is a challenge given the potentially sparse availability of subjects at a hospital or regional level.

Bai Tian et al. (“EHR phenotyping via jointly embedding medical concepts and words into a unified vector space”, BMC Medical Informatics and Decision Making, vol. 18, no. S4, 1 Dec. 2018 (2018 Dec. 1), page 13, XP055804407, DOI: 10.1186/s12911-018-0672-0) discloses using predictive modeling to tackle the heterogeneous nature of Electronic Health Record (EHR) data and to gain insight into patient phenotyping by embedding both (1) diagnostic medical codes and (2) words from clinical notes in the same continuous vector space to build connections between them. To evaluate the quality of its vector representations, Tian et al. discloses two types of experiments: (1) phenotype and treatment discovery by evaluating associations between codes and words in the vector space, and (2) predicting codes that will be assigned to a patient during a second visit by evaluating associations between codes and words in the vector space from a first visit. Tian et al. evaluated six diseases for its baseline method—acute liver failure, female breast cancer, schizophrenic disorders, conditions of the brain, depressive disorder, and HIV—none of which are as rare nor as challenging to treat as SMA.

Thus, there is a need to improve personalized selection of SMA treatments, personalized identification of treatment schedules, and the formation of subject groups for new clinical studies, so as to improve treatment efficacy for individual subjects diagnosed with SMA.

### SUMMARY

In some embodiments, a computer-implemented method is provided. The computer-implemented method can include retrieving a subject record associated with a subject and extracting a subset of the set of features included in the subject record. For example, the subject record can include a set of features characterizing the subject. The subject may have been previously diagnosed with spinal muscular atrophy (SMA). Further, each feature of the subset of the set of features may be associated with an SMA characteristic. The computer-implemented method can also include generating a partial word sequence by combining the subset of the set of features into a sequence of one or more words. Each word of the one or more words representing a feature of the subset of features. The computer-implemented method can include transforming the partial word sequence into a numerical representation using a trained word-to-vector model. The computer-implemented method can also include inputting the numerical representation of the partial word sequence into a natural language processing (NLP) model having been trained to predict a completion word or phrase for completing the partial word sequence. The computer-implemented method can further include generating, based on the completion word or phrase outputted by the NLP model, a disease progression representing a predicted progression of one or more SMA phenotypes specific to the subject over a period of time. The computer-implemented method can also include outputting an indication that the subject is predicted to exhibit the one or more SMA phenotypes included in the disease progression.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory, machine-readable storage medium and that includes instructions configured to cause one or more processors to perform part or all of one or more methods disclosed herein.

Some embodiments of the present disclosure include a system including one or more processors. In some embodiments, the system includes a non-transitory, computer-readable storage medium containing instructions which, when executed on the one or more processors, cause the one or more processors to perform part or all of one or more methods and/or part or all of one or more processes disclosed herein. Some embodiments of the present disclosure include a computer-program product tangibly embodied in a non-transitory, machine-readable storage medium, including instructions configured to cause one or more processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 illustrates a network environment in which the cloud-based application is hosted, according to some aspects of the present disclosure.

FIG. 2 is a flowchart illustrating an example of a process performed by the cloud-based application to distribute condensed subject records to user devices in association with a consult broadcast requesting assistance with treating a subject, according to some aspects of the present disclosure.

FIG. 3 is a flowchart illustrating an example of a process for monitoring the user integration of treatment-plan definitions (e.g., decision trees or treatment workflows) and automatically updating the treatment-plan definitions based on a result of the monitoring, according to some aspects of the present disclosure.

FIG. 4 is a flowchart illustrating an example of a process for recommending treatments for a subject, according to some aspects of the present disclosure.

FIG. 5 is a flowchart illustrating an example of a process for obfuscating query results to comply with data-privacy rules, according to some aspects of the present disclosure.

FIG. 6 is a flowchart illustrating an example of a process for communicating with users using bot scripts, such as a chatbot, according to some aspects of the present disclosure.

FIG. 7 is a block diagram illustrating an example of a network environment for deploying trained artificial-intelligence models to facilitate the subject-specific identification of treatments and treatment schedules, according to some aspects of the present disclosure.

FIG. 8 is a block diagram illustrating an example of a network environment for deploying a trained artificial-intelligence model to predict the disease progression for subjects diagnosed with SMA, according to some aspects of the present disclosure.

FIG. 9 is a block diagram illustrating an example of a network environment for intelligently identifying candidate subjects for new or existing clinical studies, according to some aspects of the present disclosure.

FIG. 10 is a block diagram illustrating an example of a network environment for deploying a trained artificial-intelligence model to intelligently select treatments, according to some aspects of the present disclosure.

FIG. 11 is a flowchart illustrating an example of a process for predicting the disease progression of subjects diagnosed with SMA, according to some aspects of the present disclosure.

FIG. 12 is a flowchart illustrating an example of a process for intelligently identifying candidate subjects for new or existing clinical studies, according to some aspects of the present disclosure.

FIG. 13 is a flowchart illustrating an example of a process for deploying artificial-intelligence models to facilitate the selection of treatments to perform on subjects diagnosed with SMA, according to some aspects of the present disclosure.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

In Europe, a rare disease is defined as a disease that affects less than 1 in 2,000 people. While SMA is one of the leading genetic causes of infant mortality in Europe, SMA is still a rare disease, given that approximately 10,000 individuals in Europe are affected by SMA. The population of subjects diagnosed with SMA presents several unique challenges. First, experienced physicians may not have had the opportunity to treat a subject with SMA in his or her career. Even at a hospital or regional level, the number of previously treated subjects with SMA may be limited. Without experience in diagnosing and treating subjects with SMA, correctly treating the subjects can be challenging. Due to the small numbers of subjects affected by SMA, gaining insight into the pathophysiological mechanisms of SMA and testing new treatments is limited.

Second, SMA is unique, in that the disease progression and the severity of phenotypes vary widely within each SMA Type. While SMA generally causes proximal muscles to degenerate, there are over 500 skeletal muscles that can be affected by SMA. Thus, the phenotypes of SMA and the severity of phenotypes fall on a wide spectrum across subjects. To illustrate, for example, certain subjects initially experience degeneration of pharyngeal muscles, which assist in the swallowing action, whereas, other subjects initially experience degeneration of muscles surrounding the thigh, which assists in knee extension during the walking action. Initial treatments for these two groups of subjects are very different. The subjects experiencing difficulty swallowing may be treated with a semi-solid diet by a nutritionist, whereas, the subjects experiencing difficulty walking may be provided with a wheelchair or cane as treatment to reduce fatigue on the thigh muscles. Accordingly, identifying treatments and treatment schedules is often informed in response to onset of a symptom, instead of predictively in advance of the symptom onset or before symptoms increase in severity.

Certain aspects of the present disclosure provide a cloud-based application configured with an AI system to solve SMA-specific challenges. AI-based techniques have recently been used to transform the diagnosis and treatment of rare diseases. AI techniques can be used to learn patterns and correlations across data sets of various types (e.g., structured data sets, unstructured data sets, streaming data, etc.) from different sources. For instance, even though rare diseases are characterized by a limited number of subjects who are geographically dispersed, AI techniques can be executed to facilitate the improvement of care lines and the development of new treatments for SMA.

Certain aspects of the present disclosure relate to an AI system configured to perform certain predictive functionality, such as predicting a disease progression for a particular subject with SMA, predicting candidate subject groups to evaluate or enroll in new or existing clinical studies, or predicting a contextual treatment schedule specific to a particular subject.

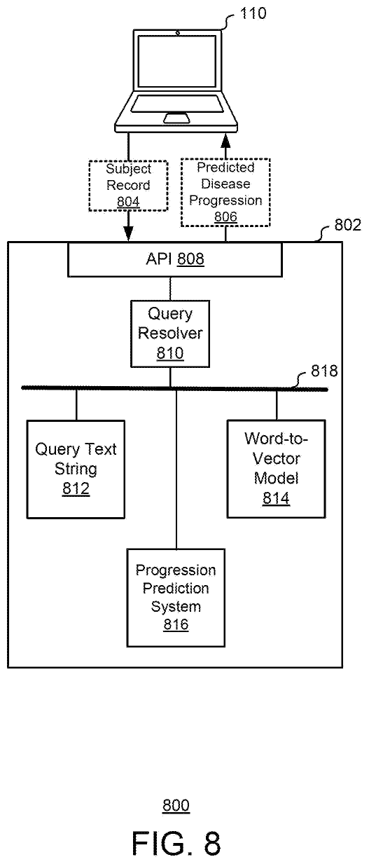

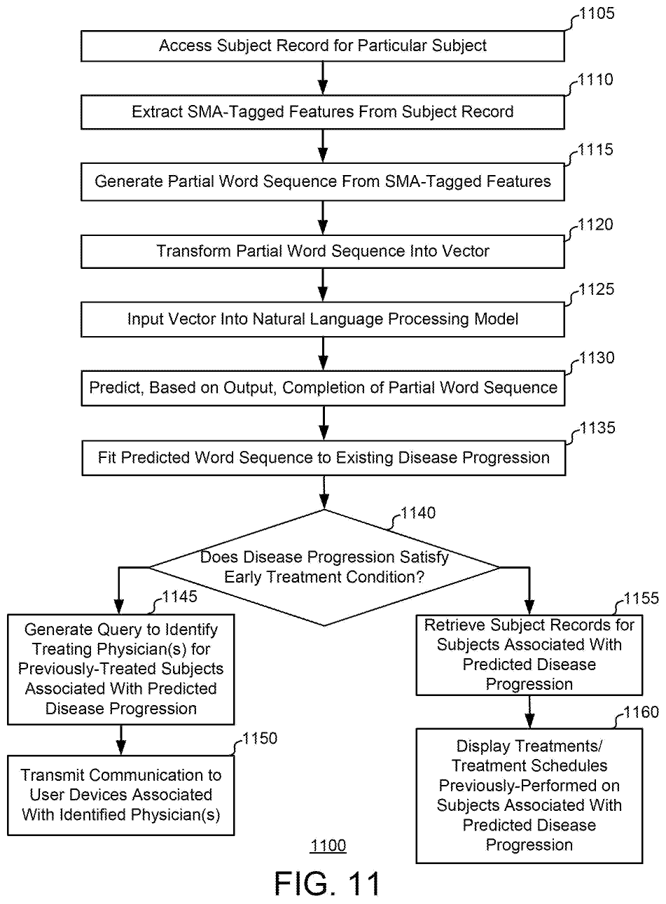

As described in greater detail with respect to FIGS. 8 and 11, certain aspects of the present disclosure relate to techniques for predicting the disease progression of a particular subject diagnosed with SMA. The AI system can train an AI model, such as a natural language processing (NLP) model or on word sequences (e.g., sentences) representing the disease progression of SMA patients. Training an NLP model on word sequences that represent the disease progression of previously-treated SMA patients enables the AI model to learn the patterns in the various combinations of words in those word sequences. The trained AI model can then receive as input the current health state of a particular subject. In some implementations, the trained AI model treats the current health state of the particular subject as a partial word sequence, and then generates a prediction of the next words that are likely to complete the partial word sequence. The predicted next words represent the predicted future disease progression for the particular subject. For example, the predicted disease progression can indicate changes in SMA-specific phenotypes, symptoms, or other disease-related events that the particular subject is predicted to exhibit over the course of the disease.

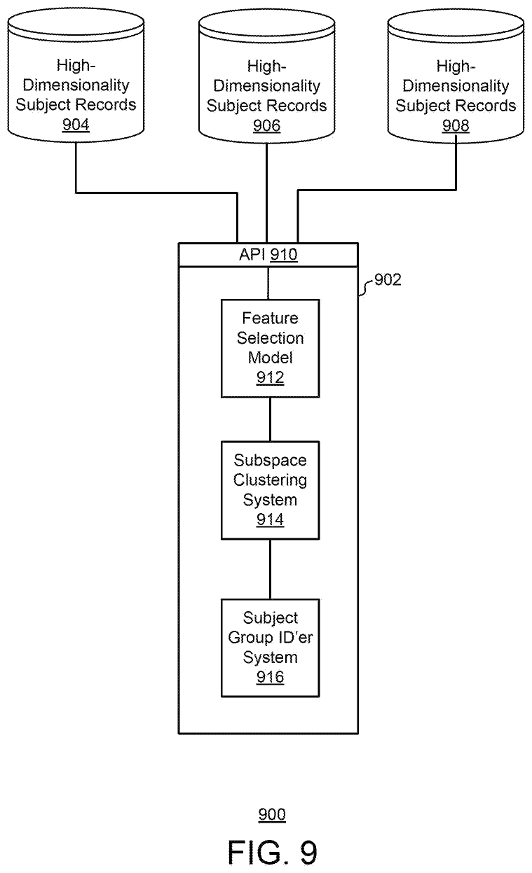

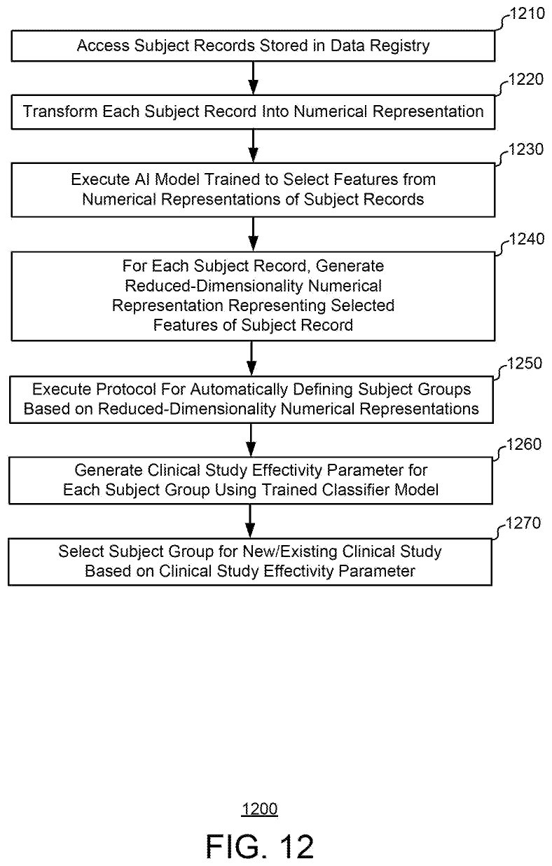

As described in greater detail with respect to FIGS. 9 and 12, certain aspects of the present disclosure relate to techniques for intelligently identifying groups of subjects who are predicted as being suitable candidates for enrollment in a new or existing clinical study. For example, a subject is a suitable candidate for enrollment in a clinical study when the treatment that is being investigated in the clinical study is predicted to be effective on the subject. In some implementations, intelligently identifying subject groups based on highly-dimensional subject records involves selectively reducing the dimensionality of subject records to improve the computational efficiency of subspace clustering of the subject records (e.g., clustering along many dimensions, not just one or two dimensions as in the case of k-means clustering). The reduced-dimensionality subject records can be used to automatically predict new groups of subjects who may be suitable candidates for a new or existing clinical study. As an illustrative example, according to certain implementations, if 40 subjects being treated for SMA at a hospital in Italy experience an improvement in motor function after a particular physical therapy, and if 17 subjects being treated for SMA at a research facility in Bogota also experience a similar improvement in motor function after the same physical therapy, an AI system can process data records corresponding to the subjects to detect latent features that are common across these two groups of subjects. Further, after the AI system detects the shared latent features, such as a particular biomarker that is shared across the subjects, then the two groups of subjects can be enrolled in an existing clinical study investigating the particular biomarker, a new clinical study can be proposed to investigate the particular biomarker if an existing clinical study does not exist.

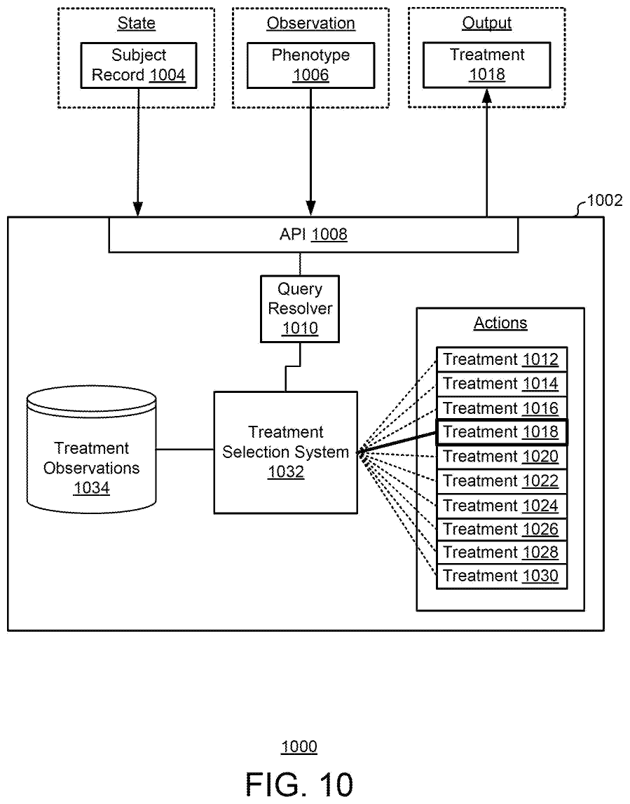

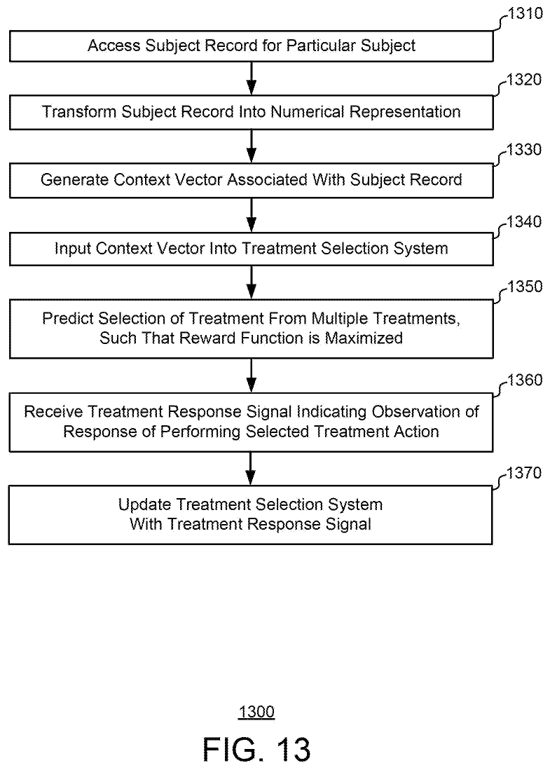

As described in greater detail with respect to FIGS. 10 and 13, certain aspects of the present disclosure relate to techniques for intelligently selecting a treatment from a group of available treatments using a treatment selection system that is trained to maximize a predefined reward function contextually based on a subject-specific data set (e.g., a subject record of a particular subject) when selecting treatments. The output of the trained AI model can be predictive of which treatment to select to achieve the highest probability of treatment efficacy, slowed disease progression, extended survival, etc., specifically for the particular subject with SMA.

An application (e.g., operating locally on a device and/or at least partly using results of computations performed at one or more remote and/or cloud servers) can be used by (for example) a subject who has SMA and/or a care provider caring for a subject that has SMA. The application can perform one or more operations disclosed herein. In some instances, one or more applications can facilitate communicate between a subject with SMA and a care provider. Such communication may (for example) facilitate alerting a care provider of an abnormal weakness in muscles supporting the spine and/or may facilitate telemedicine (e.g., which may be particularly valuable when the subject or a portion of a local society has a communicable disease, when the subject has a locomotion disability, and/or when the subject is physically far from an office of the care provider).

### II. Summary of Spinal Muscular Atrophy (SMA) Sub-Types, Diagnosis Protocol, Pertinent Medical Tests, Progression Assessment and Available Treatments

II.A. Genetic Cause of SMA

SMA is a neuromuscular disease characterized by the atrophy of skeletal muscles, which are used for voluntary movement. Subjects with SMA experience progressive degeneration of certain nerve cells located in the anterior horn of the spinal cord. These nerve cells, called spinal cord motor neurons, control the movement of muscles. The degeneration of the motor neurons weakens skeletal muscles and causes generalized weakness in subjects.

The genetic cause of SMA is a mutation in the survival motor neuron 1 (SMN1) gene located in chromosome 5. In healthy individuals, the SMN1 gene produces the survival motor neuron (SMN) protein, which is a protein necessary for the survival of motor neurons. The SMN1 gene produces the entire amount of SMN protein needed for the motor neurons to survive. In individuals affected by SMA, however, the SMN1 gene is mutated due to a deletion occurring at exon 7 or other point mutations. The deletion at exon 7 of chromosome 5 in the SMN1 gene causes a reduction in the amount of the SMN protein produced by the SMN1 gene or prevents the production of the SMN protein altogether.

SMN1 has at least one functional copy called the survival motor neuron 2 (SMN2) gene, which inefficiently produces the SMN protein that supports healthy motor neurons. For example, the SMN2 gene can produce about 10-20% of the normal level of the SMN protein needed for motor neuron survival. SMN1 and SMN2 produce the same SMN protein in different amounts because SMN1 and SMN2 are nearly identical, except for a single nucleotide at exon 7. Ultimately, however, without sufficient SMN protein, motor neurons cannot function properly and eventually shrink and die, leading to debilitating and sometimes fatal muscle weakness.

In some cases, SMA may not be a result of a mutation in the SMN1 gene at chromosome 5, but rather a mutation with another gene on another chromosome. For example, spinal muscular atrophy with respiratory distress (SMARD), which may be referred to as autosomal recessive distal spinal muscular atrophy (DSMA1), is not caused by a mutation of the SMN1 gene. Instead, SMARD is caused by mutations in the IGHMBP2 gene located on the long arm of chromosome 11. Subjects with SMARD have severe respiratory distress and muscle weakness.

While most forms of SMA, like those forms related to chromosome 5 mutations, affect proximal muscles, other forms of SMA affect distal muscles. The genetic cause of the atrophy of distal muscles may include mutations in the UBA1 gene located on the X chromosome, the DYNC1H1 gene located on chromosome 14, the TRPV4 gene located on chromosome 12, the PLEKHG5 gene located on chromosome 1, the GARS gene located on chromosome 7, and the FBXO38 gene located on chromosome 5. The UBA1 gene listed above can cause X-linked SMA (e.g., XL-SMA or SMAX2). X-linked SMA is similar to SMA Type-I, however, in X-linked SMA, joints may be also affected. Other symptoms of X-linked SMA may include hypotonia, lack of reaction to stimuli, and congenital contractures.

II.B. Types of SMA

SMA generally manifests early in a subject's life and is the leading genetic cause of death in infants and toddlers, affecting approximately one in 10,000 births. Roughly one in 40-60 people are carriers of the SMN1 genetic mutation that causes SMA. SMA is inherited in an autosomal recessive pattern, with no significant differences in occurrence rate among ethnic groups. There is roughly a 25% chance that a newborn will have SMA if both parents are carriers of the SMN1 genetic mutation.

There are four primary types of SMA: Type-I, -II, -III, and -IV, with an additional very rare and severe Type-0. The types of SMA differ based on the age that symptoms begin and highest attained milestone in motor development.

II.B.1. SMA Type-0

SMA Type-0 is a very rare, prenatal form of the SMA disease. SMA Type-0 is detectable in utero because a subject fetus presents severe SMA symptoms before birth. For example, a subject fetus diagnosed with Type-0 presented generalized osteopenia in the lower limbs.

SMA Type-0 generally has a fatal prognosis with symptom onset being intrauterine, leading to hypotonia, facial weakness, and may lead to death within the first few weeks to three months of the subject infant's life. Homozygous mutations of the SMN1 gene may be a cause of SMA Type-0. Available diagnostic tests can show the absence of SMN1 exon 7, demonstrating the homozygous deletion of the SMN1 gene.

Further, SMA Type-0 subjects have presented with reduced muscle movement in utero, severe asphyxia, profound hypotonia, respiratory insufficiency at birth and a need for resuscitation and ventilator support. Additionally, an alert look on the subject has been uniformly observed.

II.B.2. SMA Type-I

SMA Type-I, also known as Werdnig-Hoffman disease, usually manifests in the first few months of life. The most severe form of SMA Type-I has a quick and unexpected onset. As the disease progresses, rapid motor neuron death causes inefficiency of major bodily organs, especially of the respiratory system. Pneumonia-induced respiratory failure is the most frequent cause of death. If untreated and without respiratory support, infants diagnosed with SMA Type-I usually do not survive past two years of age. With proper respiratory support, those with milder SMA Type-I phenotypes can survive into adolescence and adulthood.

II.B.3. SMA Type-II

SMA Type-II, also known as Dubowitz disease, affects individuals who were able to maintain a sitting position at some point in their lives, but never learned to walk unsupported. The onset of SMA Type-II usually occurs between six and 18 months of life, with progression varying greatly, as some children gradually grow weaker while others remain relatively stable. Scoliosis is usually present in these children, and spinal correction can improve respiration. Although life expectancy is reduced, most people with SMA Type-II live well into adulthood.

II.B.4. SMA Type-III

SMA Type-III, also known as Kugelberg-Welander disease, is a juvenile form of the disease that usually appears after 12 months of age. Patients with SMA Type-III are characterized as having the ability to walk without support for at least some time in their lives, even if this ability was later lost. Respiratory involvement is less frequent in this form of the disease and life expectancy is normal or near normal.

II.B.5. SMA Type-IV

SMA 4, is an adult-onset form of the disease that usually manifests after the age of 30, with gradual weakening of leg muscles and frequently requires the subject to use mobility aids. Other complications are rare and life expectancy is normal.

II.B.6. Severity of Phenotypes Across SMA Sub-Types

Every subject who has SMA has at least one SMN2 copy of the SMN1 gene. For a given subject, the number of SMN2 copies influences the prognosis of the subject because the number of SMN2 gene copies a subject has is correlated with the severity of SMA phenotypes. For instance, the greater the number of SMN2 gene copies a subject has, the milder the symptoms and the later the onset of symptoms. The presence of greater numbers of SMN2 gene copies, the more functional SMN protein is available, and thus, the later the onset of disease symptoms due to the increased survival of motor neurons.

The severity of SMA across SMA sub-types is thus influenced by the number of SMN2 copies a subject has. For example, about 70% of SMA-I subjects carry two SMN2 copies and 82% of SMA-II subjects have three SMN2 copies. However, subjects with SMA-III overwhelmingly have a minimum of three to four SMN2 copies. The SMN1 gene produces roughly 100% of a full-length mRNA of the SMN protein. The SMN2 gene, however, produces transcripts of the SMN protein that lack exon 7. As a result, about 10% of the SMN protein encoded by the SMN2 gene are correctly spliced and encode a protein identical to SMN1. Accordingly, more SMN2 copies reduces the deficiencies of the SMN protein.

II.C. Diagnosis of SMA Sub-Types

Diagnosing SMA involves a series of steps. Initially, a physician may conduct an in-office physical examination and review of a subject's family history. Certain non-invasive tests may be performed to determine whether a genetic test should be performed. The non-invasive tests assist the physician in distinguishing SMA from other neuromuscular conditions (e.g., muscular dystrophy). For example, if the subject is ambulatory, the physician may perform motor function tests, such as the Hammersmith Functional Motor Scale-Expanded (HFMSE) test and the 6-Minute Walking Test (6MWT). The HFMSE and 6MWT motor function tests are highly correlated with predicting SMA phenotype severity. Further, the physician may assess for muscle weakness and hypotonia, which are early indications of the existence of motor function issues associated with SMA. Other assessments may include evaluating the subject for a history of motor function difficulties, loss of motor skills, proximal muscle weakness, the absence of reflexes, tongue fasciculations, and other indicators of the degeneration of motor neurons. Further, the most common symptoms that prompt diagnostic genetic testing for SMA include progressive bilateral muscle weakness (usually in the upper arms and legs), bell-shaped chest, and hypotonia associated with absent reflexes. These symptoms are more prevalent and often severe in SMA Type-0 and SMA Type-I subjects.

A blood test for creatine kinase may indicate a likelihood for SMA because creatine kinase is an enzyme that is excreted from deteriorating muscles. While creatine kinase enzyme levels are above a threshold level for multiple neuromuscular diseases, the results of such a blood test are nonetheless informative for a physician diagnosing a subject. While levels of the creatine kinase enzyme can be normal for certain subjects with SMA Type-I, the creatine kinese levels can be informative for diagnosing SMA Types-II and -III.

If early assessments of a subject's symptoms indicate motor function issues associated with SMA, a genetic test may be performed for the subject. A diagnosis of SMA can only be confirmed through genetic testing, for example, by detecting a bi-allelic deletion of exon 7 or other point mutations in the SMN1 gene. Other methods are available for genetic testing, but multiplex ligation-dependent probe amplification (MLPA) is often used, as this method also allows detection of the number of SMN2 gene copies in the subject. Several MLPA genetic testing kits are commercially available, for example, Asuragen's Amplidex PCT/CE SMN1/2 Kit, and Prevention Genetics' Spinal Muscular Atrophy via MLPA of SMN1 and SMN2 test.

In addition or in lieu of genetic testing, an electromyography (EMG) test may be performed. The EMG test measures the electrical activity of a muscle or group of muscles, a muscle biopsy and/or a creatine kinase (CPK) test can be also be used to diagnose SMA, as well as distinguish the diagnosis from other types of neuromuscular disease, if necessary.

In addition to diagnostic testing of symptomatic individuals, prenatal genetic testing, and newborn screening can be performed to diagnose the early stages of severe forms of SMA, for example, SMA Type-0 and SMA Type-I.

II.D. Newborn Screening for SMA

Newborn screening for SMA can be a part of routine screening for newborns during the first few days of an infant's life. Newborn screening for SMA is a genetic test on a newborn's blood. The genetic test includes evaluating the newborn subject's blood sample for abnormalities associated with the SMN1 gene. While a genetic test on blood is invasive, the newborn screening for SMA uses the same blood samples already collected for the screening of other disorders. When the results of a newborn's blood sample analysis indicates that the newborn is missing portions of the SMN1 gene located at chromosome 5, the newborn is likely to have or at high risk of having SMA. Additional tests can be performed to determine whether the infant subject has SMA and, if so, to identify target treatment for the infant subject.

According to certain studies, screening all newborns in the United States for SMA, for example, would likely detect about 364 newborns with the disorder each year. Further, widespread newborn screening could prevent roughly 50 newborns from needing a ventilator and about 30 deaths due to SMA Type-I. Additionally, newborn screenings are critical because early treatment relative to symptom onset is more effective than late treatment relative to symptom onset.

Newborn screening programs can also be used to identify presymptomatic newborns. In many cases, if therapeutic treatment is initiated before symptom onset, the treatment can prevent irreversible motor neuron damage. Homozygous mutations of the SMN1 gene have been shown to be accurately detectable in blood samples from newborns, which proves the screening of newborns for SMA using blood samples taken on the day of birth to be a useful screening approach.

Newborn screening for SMA has limitations. For instance, point mutations in the SMN1 gene for certain subjects is difficult to detect. Prenatal screen and treatment may be suitable for certain subjects. In murine cell models, for instance, the SMN protein assists neuronal differentiation and the formation of the neuromuscular junctions in utero. The SMN protein is also involved in neurodevelopment and synaptogenesis. Thus, prenatal screening for SMA for certain subjects and potentially the prenatal or neonatal treatment of subjects diagnosed with SMA Type-0 may be feasible and useful for early detection and treatment. For certain subjects, prenatal screening for SMA may be feasible; especially given fetal gene replacement therapies, such as administering adeno-associated viruses (AAV) that can infect and deliver the SMN1 gene to a subject's cells. Additionally, for certain subjects, chorionic villus sampling or amniocentesis can be performed at 10-14 or 15-20 weeks of gestation. This sampling has been shown to identify the likelihood or risk of a fetus having SMA. Prenatal screening, however, also has its own challenges and limitations. Prenatal screening is invasive and could create risks for the mother and fetus. Noninvasive prenatal screening for SMA is possible. In certain studies, fetal trophoblastic cells or cell-free fetal DNA were isolated from the mother's blood sample and evaluated to detect SMA.

II.E. Clinical Symptoms of SMA

Although symptoms vary depending on the type of SMA, the stage of the disease, and individual factors, the signs and symptoms of SMA include delayed gross motor skills, difficulty standing, sitting or walking, adopting a frog-leg position when sitting, areflexia (particularly in the extremities), overall muscle weakness, poor muscle tone, limpness, tendency to flop, loss of strength in respiratory muscles, gastrointestinal issues, cough, accumulation of secretions in the lungs or throat, respiratory distress, a bell-shaped torso, scoliosis, twitching (fasciculations) of the tongue, difficult sucking or swallowing, and poor feeding.

II.F. SMA Treatments

Treatment of SMA varies based on the severity and type. In the most severe forms (SMA 0 and SMA 1), individuals have the greatest muscle weakness, requiring prompt intervention. In contrast, individuals who have SMA 4, or adult onset SMA, may not require treatment until much later in life. Treatment of severe SMA is often difficult, as timelines for diagnosis and treatment can be very short due to the patient's age or current health status. Since SMA is a rapidly progressive disease that affects the muscles involved in swallowing, breathing, and feeding, it can become life-threatening very quickly. Therefore, early diagnosis and aggressive treatment of individuals with SMA 0 and SMA 1 are critical.

Currently, nusinersen (Spinraza®), an antisense oligonucleotide that modifies alternative splicing of the SMN2 gene, is used to treat SMA. SMN2 splicing modulation forces the SMN2 gene to produce increased amounts of a full-length SMN protein. Nusinersen is administered directly to the central nervous system, via intrathecal injection, to prolong survival and improve motor function in infants with SMA. Other SMN2 gene splice modulators that increase the availability of SMN protein in motor neurons include orally administered small molecules such as Branaplam (LMI070, NVS-SM1) and Evrysdi (risdiplam, RG7916, R07034067) (F. Hoffman-La Roche AG). Evrysdi can be administered to treat Types 1, 2 and 3 SMA, in adults and children two months of age and older. Zolgensma® (onasemnogene abeparvovec) is a gene therapy which uses self-complementary, adeno-associated virus type 9 (scAAV-9) as a vector to deliver the SMN1 transgene. This treatment was approved in the United States as an intravenous formulation to treat those younger than two years of age.

Other treatments include olesoxime (F. Hoffman-La Roche AG), a neuroprotective compound, and albuterol, an SMN2 gene activator.

Depending on the severity and type of SMA, respiratory support is often used to manage SMA. In some cases, respiratory issues are caused by accumulating airway secretions. Manual or mechanical chest physiotherapy with postural drainage can be used to clear secretions. In addition, a manual or mechanical cough assistance device, or a non-invasive ventilation (BiPAP) can be used. In more severe cases, a tracheostomy can be performed.

Nutritional support can also be essential as feeding, jaw opening, chewing, and swallowing can be compromised due to SMA. Other nutritional issues include food not passing through the stomach quickly enough, gastric reflux, constipation, vomiting, and bloating. Therefore, SMA patients, particularly SMA 1 patients, sometimes require a feeding tube or gastrostomy. Metabolic abnormalities resulting from SMA, impair β-oxidation of fatty acids in muscles and can lead to organic acidemia and consequent muscle damage, especially when fasting. Individuals with SMA, especially those with more severe forms of the disease, should choose softer foods to avoid aspiration, reduce fat intake and avoid prolonged fasting.

Management of SMA can also include treatment of orthopedic issues resulting from disease progression. Skeletal problems associated with weak muscles in SMA include tight joints, hip dislocations, spinal deformity, osteopenia, an increase risk of fractures, and pain. Weak muscles can lead to development of kyphosis, scoliosis, and/or joint contracture. Spine fusion is sometimes performed in people with SMA I/II, to relieve the pressure of a deformed spine on the lungs. Furthermore, mobility devices (e.g., wheelchair, crutches, cane, walker), range of motion exercises, and bone strengthening can help prevent orthopedic complications. Occupational therapy and physical therapy are also helpful. Orthotic devices, for example, ankle foot orthoses, and thoracic lumbar sacral orthoses can also be used to support the body and to aid in walking.

In recent years, survival of SMA patients has increased with available drug treatments as well as aggressive respiratory, orthopedic, and nutritional support.

II.F.1 Treatment Window for Effective Restoration of SMN Protein Levels

Early treatment of SMA is critical. For example, studies have shown that pre-emptive treatment of SMA subjects before or around the time of symptom onset can increase motor function and quality of life. Restoring SMN protein levels early, for example, in some cases, between days 1-3 of life, is more effective at increasing motor function than restoring SMN protein levels after day 5 of life.

II.G. Disease Progression for SMA

The various types of SMA are degenerative. SMA may present differently across the various types of SMA.

II.G.1. Disease Progression for SMA Type-I

For a given SMA Type, the proximal muscles of a subject degenerate first. Distal muscles are then strained given the degeneration of the subject's proximal muscles. For example, a subject's thigh muscles may weaken first, which strains the subject's foot muscles. For most subjects with SMA, the hands maintain strength the longest, such that daily tasks (e.g., using a computer) are performable even as the disease progresses.

SMA can lead to scoliosis (e.g., an “S”-shaped curve in the spine) because the subject's muscles that support the spine weaken over time. Subjects with scoliosis may exhibit uneven shoulders and hips, or a hip or a shoulder on one side may be larger than the corresponding hip or shoulder on the other side of the subject. Given the weakening of the muscles that support the spine, subjects with SMA often experience respiratory issues that could be life threatening.

For children with SMA Type-I, the disease is also called Werdnig-Hoffmann disease, which is a severe form of SMA. Werdnig-Hoffmann disease can be diagnosed at birth up to 6 months of life. SMA Type-I in certain children can result in significant muscle weakness, such that the children cannot sit or stand on their own accord. Children can also experience difficulty sucking or swallowing, which can cause malnutrition.

II.G.2. Disease Progression for SMA Type-II

Disease progression for children with SMA Type-II varies significantly. Some children can be in a seated position on their own early in life, but not later, such as in their teens. Further, Type-II subjects who are ambulatory may experience difficulty walking a few feet unassisted. Fingers may begin trembling. Tendon reflexes can also be diminished. By mid-teens or later, SMA Type-II subjects typically cannot sit independently. As with other SMA Types, subjects with Type-II often experience muscle weakness in muscles near the spine, causing potentially life-threatening breathing issues.

II.G.3. Disease Progression for SMA Type-III

SMA Type-III, also known as Kugelberg-Welander syndrome, can be diagnosed at 18 months of life. Symptoms may be detected earlier. For example, children with Type-III can walk, but may experience difficult climbing or walking up stairs. Children also experience difficulty sitting up from a supine position. Further, similar to other forms of SMA, Type-III subjects may likely exhibit issues with breathing or other respiratory issues as the muscles that support their spines degenerate. In some subjects, SMA Type-III can be diagnosed between 20-30 years of age, and in these situations, disease progression may be slow. Adults with SMA Type-III are typically ambulatory, however, and as they age, walking will become more difficult.

### III. Overview of Cloud-Based Network Architecture for Deploying Intelligent Functionality

Techniques relate to configuring a server to execute code that enables a user (e.g., a physician) of an entity to execute machine-learning or artificial-intelligence techniques using subject records. Subject records include a complex combination of data elements that characterize subjects. As an illustrative example, a subject record may include a combination of thousands of data fields. Some data fields may contain fixed non-numerical values (e.g., a subject's ethnicity), other data fields may contain unstructured text data (e.g., notes prepared by a physician), other data fields may include a time-variant series of collected measurements (e.g., glycosylated hemoglobin measurements taken two to four times a year), and other data fields may include images (e.g., MRI of a subject's brain). The complexity and variance of data types and formats in subject records make processing subject records technically challenging, if not impossible, because machine-learning and artificial-intelligence models are often configured to process data in numerical or vector form. In light of this objective technical problem, certain aspects and features of the present disclosure relate to transforming subject records into transformed representations, such as vector representations, that characterize the various data elements of the subject records.

Techniques relate to transforming the non-numerical values included in subject records into numerical representations (e.g., feature vectors) that can be inputted into machine-learning or artificial-intelligence models to generate predictive outputs. The server executing the code provides a technical effect, which solves the objective technical problem by transforming the subject records into transformed representations that are consumable by machine-learning or artificial-intelligence models. “Consumable” may refer to data that is in a format or form, which machine-learning or artificial-intelligence models are configured to process to generate predictive outputs. Machine-learning or artificial-intelligence models are not be configured to process subject records (as they exist in their stored state in the data registries) due to the complex combinations of data elements in multiple different data formats and data types contained in each individual subject record. To illustrate, for a given subject record, a data element may include a longitudinal sequence of events (e.g., an immunization record), another data element may include measurements taken from a subject (e.g., vitals), yet another data element may include text entered by the user (e.g., notes taken by the physician), and yet another data element may be an image (e.g., an X-ray). A limited or simplistic analysis may be performed on subject records (before any transformations), such as grouping subjects based on a value of a data element (e.g., age group). However, the limited or simplistic analysis becomes problematic or infeasible as the complexity and size of subject records reaches a Big-Data scale. To process and extract analytical assessments from the subject records at a Big-Data scale, machine-learning or artificial-intelligence techniques can be used for data mining the subject records. Machine-learning or artificial intelligence models, however, are configured to receive numerical or vector inputs. For example, clustering operations, such as k-means clustering, are configured to receive vectors as inputs. Thus, to perform the clustering operation on subject records, the present disclosure provides a technical effect, which solves the objective technical problem, by transforming the subject records into transformed representations, such as numerical vector representations, that are consumable by machine-learning or artificial-intelligence models. An intelligent analysis can be performed on subject records in their transformed representation state. Non-limiting examples of intelligent analysis (performed upon the server executing code) may include automatically detecting subject groups using clustering techniques, generating outputs predictive of certain outcomes based on the values of data elements in subject records, and identifying existing subject records that are similar to a given or new subject record.

To illustrate and only as a non-limiting example, a subject record of a subject includes four data elements. The first data element contains a unique code that represents a diagnosis of a condition. The second data element contains an MRI of the subject's brain. The third data element contains a time-variant series of measurements, such as blood pressure readings, over the course of one year. The fourth data element contains unstructured notes, for example, notes of a condition detected by examining or running one or more tests. According to certain implementations, each of the first data element, the second data element, the third data element, and the fourth data element may be transformed into a transformed representation (e.g., a vector). The techniques used for transforming the values contained within the four data elements may depend on the type of data contained in a data element. For the first data element, for example, the unique code that represents a diagnosis can be represented as a fixed length vector, such that the size of the vector is determined by a size of a vocabulary of codes, and that each code in the vocabulary is represented by a vector element of the fixed length vector. The one or more unique codes contained within the first data element may be compared with the vocabulary of codes. If a unique code matches a code of the vocabulary, then a “1” may be assigned to the vector element at the position of the vector that corresponds to the unique code and a “0” may be assigned to all remaining vector elements of the vector. In light of the above, a first vector may be generated to represent the value of the first data element. As another example, for the second data element, a latent-space representation of the image may be generated using a trained auto-encoder neural network. The latent-space representation of the input image may be a reduced-dimensionality version of the input image. The trained auto-encoder neural network may include two models: an encoder model and a decoder model. The encoder model may be trained to extract a subset of salient features from the set of features detected within the image. A salient feature (e.g., a key point) may be a region of high intensity within the image (e.g., an edge of an object). The output of the encoder model may be a latent-space representation of the input image. The latent-space representation may be outputted by a hidden layer of the trained auto-encoder model, and thus, the latent-space representation may only be interpretable by the server. The decoder model may be trained to reconstruct the original input image from the extracted subset of salient features. The output of the encoder model may be used as the feature vector that represents the pixel values of the image included in the second data element. In light of the above, a second vector (e.g., the latent-space representation) may be generated to represent the image contained in the second data element. As another example, for the third data element, the time-variant sequence of measurements can be represented numerically. In some implementations, the time-variant sequence can be represented by a total of the instances a measurement was taken from a subject. In other implementations, the time-variant sequence can be represented numerically using an average, mean, or median of the values of the measurements taken across the instances of measurements that occurred during a time period (e.g., one year). In other implementations, a frequency of measurements can be calculated and used to numerically represent the time-variant sequence of measurements. In light of the above, a third vector may be generated to represent the time-variant sequence of values contained within the third data element. As yet another example, for the fourth data element, the notes inputted by the user may be processed and vectorized using any number of natural language processing (NLP) text vectorization techniques. In some implementations, a word-to-vector machine-learning model, such as a Word2Vec model, may be executed to transform the notes contained in the fourth data element into a single vector representation. In other implementations, a convolutional neural network may be trained to detect words or numbers within text that indicate symptoms, treatments, or diagnoses from the notes contained in the fourth data element. In light of the above, a fourth vector may be generated to represent the text of the notes contained of the fourth data element as a vector representation. Thus, the final feature vector that represents the entire subject record may be a vector of vectors, including a concatenation of the first vector, the second vector, the third vector, and the fourth vector. In other examples, an average of the first vector, the second vector, the third vector, and the fourth vector may be used to numerically represent the entire subject record. Other combinations of the first vector, second vector, third vector, and fourth vector may be used to generate the final feature vector that numerically represents the entire subject record.

In some implementations, instead of generating a vector to numerically represent each data element of a subject record, techniques may be executed to reduce the dimensionality of the subject record by identifying and selecting a subset of data elements from the set of data elements. The subset of data elements may represent the “important” data elements, where “importance” of a data element is determined based on a prediction using feature extraction techniques, such as Singular Value Decomposition (SVD). For example, transforming a subject record into a transformed representation that is consumable by machine-learning and artificial-intelligence models may include performing one or more feature extraction techniques on the non-numerical values included in the data elements of a subject record to generate a feature vector that numerically represents a decomposed version of the non-numerical values. In some implementations, feature extraction techniques may include, for example, reducing the dimensionality of a set of data elements of a subject record (e.g., each data element representing a feature or dimension of a subject) into an optimal subset of features that can be used to, for example, predict an outcome or event. Reducing the dimensionality of the set of data elements may include reducing N data elements into a subset of M elements, where M is smaller than N. In these implementations, each element of the subset of M elements may be transformed into a numerical value. In some implementations, a feature vector may be generated to represent the N data elements of a subject record. The feature vector may include a vector for each data element of the set of data elements. For example, the feature vector may be a numerical representation of the complex combinations of data elements of a subject record. Each non-numerical value in a data element of a subject record can be vectorized to generate a representative vector. The vectors representing the set of data elements in a subject record may be concatenated or combined (e.g., as an average or weighted average) to generate the feature vector that numerically characterizes the entire set of data elements of the subject record. The feature vector is consumable by a trained machine-learning or artificial-intelligence model. Once the feature vector for a subject record is generated, the subject record can be evaluated individually or in groups of other subject records using machine-learning and artificial-intelligence techniques. After the feature vector that represents each subject record has been generated and stored, the feature vectors of the subject records stored in a central data store can be inputted into machine-learning or artificial-intelligence models or other enhanced analyses can be performed on the numerical representations of the subject records. For example, two different subject records can be compared with respect to one or more dimensions. A dimension may represent a feature or data element of a subject record, along which a comparison between two or more subject records is made. To illustrate, a data element of a first subject record contains text inputted by a first user (e.g., doctor) describing symptoms of a first subject. The text (e.g., the value of the data element of the first subject record) can be vectorized using the text vectorization techniques (e.g., Word2Vec) described above to generate a first vector to numerically represent the text associated with the data element. The text vectorization technique may generate an N-dimensional word vector for each word included in the text. The matching data element of a second subject record (e.g., the data element of another subject record that also contains text inputted by a physician describing symptoms of another subject) may contain text inputted by a second user describing the symptoms of a second subject. The text (e.g., the value of the data element of the second subject record) can be vectorized using the text vectorization techniques described above to generate a second vector (e.g., an N-dimension word vector) to represent the text associated with the data element. A server may compare the first vector with the second vector in a Euclidean or cosine space to quantify a similarity or dissimilarity between the first subject record and the second subject record at least with respect to the dimension of a subject's presentation of symptoms. If the first vector and the second vector are near each other (or within a threshold distance) in the Euclidean space (e.g., if the Euclidean distance between the first vector and the second vector is small), then the symptoms experienced by the first subject (as described in the text of the data element) are likely similar to the symptoms experienced by the second subject (as described in the text of the data elements). However, if the Euclidean distance between the first vector and the second vector is large or above the threshold distance (e.g., or if the Euclidean distance is above a threshold), then the symptoms experienced by the first subject can be predicted to be different from the symptoms experienced by the second subject.

In some implementations, a server may be configured to execute an application that enables a user of an entity to build data registries that serve to store subject records for subsequent processing. The data of a subject record may include unstructured data, such as electronic copies of physician notes and/or responses to open-ended questions. The unstructured data can be ingested into the data registries by mapping portions of the unstructured data to fixed parts (e.g., data elements) of structured data records. The structure of the structured data records may be defined using (for example) specifications from a module that corresponds to a particular use case (e.g., particular disease, particular trial, etc.). For example, each word of the unstructured note data (e.g., text) may be transformed into a numerical representation and the various numerical representations associated with the unstructured note data can be decomposed (e.g., using SVD) to detect words describing a particular set of symptoms that the subject has exhibited. The decomposition of the numerical representations of the unstructured note data may remove non-informative words, such as “and,” “the,” “or,” and so on. The remaining words represent the particular set of symptoms. Some portions of the note data may be irrelevant with regard to data elements in the structured data and/or may be more or less specific than data contained in data elements. In some instances, various mapping (e.g., mapping a “poor balance” symptom to a “neurological” symptom), natural-language-processing, or interface-based approach (e.g., that requests new information from a user) can be used to obtain structured data records. An interface may also be used to receive input that identifies new information about a new or existing subject, and the interface may include input components and selection options that map to a structure of data records.

Further, techniques relate to configuring a cloud-based application to transform non-numerical values contained in data elements of subject records into numerical representations, so that the cloud-based application can execute intelligent analytical functionality using the numerical representations (e.g., the transformed representations) of the subject records stored in the data registries. The transformation of non-numerical values of data elements of subject records to numerical representations may be dependent on the type of data contained in a data element. For example, for data elements that include text, such as notes taken by a user, the text may be transformed into numerical representations of the text using natural language processing techniques, such as Word2Vec or other text vectorization techniques. As another example, for data elements that include images (e.g., MRIs) or image frames of a video (e.g., a video of an ultrasound), each image or image frame may be transformed into a numerical representation (e.g., vector) using a trained auto-encoder neural network, which is trained to generate a latent-space representation of an input image. The condensed representation of the input image (e.g., the latent-space representation) may serve as the vector that numerically represents the input image. As yet another example, for data elements that include a time-variant sequence of information (e.g., events occurring over a period of time), the time-variant information can be represented as a numerical representation using several exemplary transformations. In some instances, the count of events may be used as the vector representing the time-variant information. In other instances, the frequency or rate of events occurring (e.g., per week, per month, per year, etc.) may be used as the vector representing the time-variant information. In still other instances, an average or combination of the measurement values associated with each event in the time-variant information can be used as the vector representing the time-variant information. The present disclosure is not limited to these examples, and thus, other numerical representations of time-variant information can be used as the vector that represents the numerical representation. Intelligent analytical functionality may be performed by executing trained machine-learning or artificial-intelligence models using data records. The model outputs may be used to indicate certain analytics extracted from the data records.

In some instances, transmission of data from a subject record may be provided to develop a treatment plan for an individual subject. For example, subject-record information (e.g., that complies with data-privacy restrictions via, for example, select omission and/or obscuring of data) may be broadcast and/or transmitted to a select group of user devices. For example, a broadcast may be transmitted to user devices associated with similar data records in response to input from the user corresponding to a request to initiate a consult with a user associated with a similar subject. If a user receiving the broadcast accepts a consultation request (via provision of corresponding input), a secure data channel may be established between the user and potentially more of the subject record may be shared (e.g., while conforming to data-privacy restrictions applicable to the two users). Subject records that are similar to a given subject may be identified by performing a nearest-neighbor technique using the vector representations of two or more subject records. Nearest neighbor techniques may be performed by comparing vectors of individual data elements across multiple subject records (e.g., the nearest neighbor may be determined in association with a dimension or feature of the subject records). Alternatively, the nearest neighbor techniques may be performed by comparing the overall vector that characterizes the entire subject record with the overall vector that characterizes another entire subject record. An overall vector may be a concatenation of individual vectors representing the values of the data elements, or may be an average or combination of the individual vectors representing the values of the data elements.

As another example, one or more processed data records may be returned in response to a query for subject records matching particular constraints. In some instances, a first user may submit a query that identifies a first subject record. The query may correspond to a request to identify other subject records that are similar to the first subject record. A server may transform the first subject record into a transformed representation using certain transformation techniques, discussed above and herein. Alternatively, the transformed representation of the first subject record may have previously been generated and stored in a database. Regardless of whether the transformed representation of the first subject record is generated before or after the query is received, transforming the first subject record into a transformed representation of the first subject record may include generating a vectorization of one or more non-numerical values of data elements of the first subject record. Vectorizing the one or more non-numerical values contained within the first subject record may include generating a numerical vector representation for each value (e.g., for non-numerical text, such as notes) included in each data element of the first subject record. The various vector representations may be concatenated or otherwise combined (e.g., an average may be computed) to generate the feature vector that represents the entire first subject record. The vector representation that numerically represents the first subject record may be compared in a domain space (e.g., Euclidean space or cosine space) to vector representations of other subject records. When the Euclidean distance, for example, between two vector representations is within a threshold distance, then the two subject records associated with the two vector representations may be interpreted (e.g., by a server) as being similar at least with respect to one or more dimensions.

For each data element in a subject record, the technique used to generate the vector representation of the value associated with the data element may depend on the type of data associated with the data element. In some examples, the data element of a subject record may be associated with one or more images, such as X-rays of the subject. Feature extraction techniques may be executed to generate a vector representation of each image associated with the data element. For example, a server may be configured to execute a trained auto-encoder neural network to generate a reduced-dimensionality version of the image. The trained auto-encoder neural network may include two models: an encoder model and a decoder model. The encoder model may be trained to extract a subset of salient features from the set of features detected within the image. A salient feature (e.g., a keypoint) may be a region of high intensity within the image (e.g., an edge of an object). The output of the encoder model may be a latent-space representation of the input image. The latent-space representation may be outputted by a hidden layer of the trained auto-encoder model, and thus, the latent-space representation may only be interpretable by the server. The subset of salient features of the latent-space representation that characterizes the subject record can be compared against the subset of salient features of the latent-space representation that characterizes another subject record to yield certain analytical insights. The decoder model may be trained to reconstruct the original input image from the extract subset of salient features. The output of the encoder model may be the vector representation of the data element associated with the image included the subject record. In other examples, keypoint matching techniques may be executed to match keypoints of an image contained in a data element of a first subject record to keypoints of another image contained in a data element of a second subject record. The vector representation (e.g., the latent-space representation) of the input image is consumable by machine-learning or artificial-intelligence models, and thus, two different subject records (each including an image) may be compared against each other to determine a similarity or a dissimilarity between the two different subject records.

To illustrate and only as a non-limiting example, a magnetic resonance image (MRI) of a subject's brain is captured. The MRI is stored in the subject record associated with the subject. The server is configured to generate a transformed representation, such as a vector representation, of the MRI contained in the subject record using feature extraction techniques, such as keypoint detection, auto-encoding to latent-space representations, SVD, and other suitable computer-vision techniques. The vector representation of the data element that contains the MRI is concatenated or otherwise combined (e.g., averaged) with the vector representations of each remaining data element of the set of data elements to generate the feature vector that characterizes the entire subject record. A user may access an application to query a database of other subject records to retrieve a set of subset other subject records that contain MRIs that are similar to the MRI of the subject's brain. Identifying other subject records that are similar to the subject record (at least with respect to similarity between MRIs) may involve calculating the k-nearest neighbors of the subject record. For example, the transformed representation may be plotted (visually or internally by a computing system) on a domain space, such as a Euclidean space or cosine space. The transformed representation of each other subject record may also be plotted (visually or internally by a computing system). A nearest-neighbor technique may be executed to compare the vector representation of the subject record with the vector representations of the other subject records to identify the k nearest neighbors to the subject vector. The k nearest neighbors that are identified may be predicted to have MRIs that are similar to the MRI of the subject's brain. Each other subject record that is identified as a nearest neighbor may be identified and retrieved for further evaluation or processing using the application.

In some implementations, a computing system may perform a data-processing technique (e.g., nearest-neighbor technique) to identify similar subject records. Various data elements may be differentially weighted in this search (e.g., in accordance with predefined data element weightings, user input that indicates an importance of matching various data elements, and/or a prevalence of particular data element values across a subject record set). When searching across a set of records for potential matches, some records may lack values for various data elements. In these cases, it may be determined that (for example) the data element values do not match and/or the data element may be unweighted when evaluating the potential match. Handling of the missing-value may depend on a distribution of values for the data element across the set of records and/or the value for the data element in the query.

Further, some techniques relate to defining and using a set of rules used to identify potential treatment regimens for a subject given a set of symptoms identified in the subject record. To illustrate, a target subject record may represent a target subject who recently experienced three symptoms: an upper respiratory infection, a fever, and a sore throat. The three symptoms may be written as text within a data element of the target subject record (e.g., the separation between words being marked by a tag, such as a semicolon). A server, such as cloud server 135, may individually input the text “upper respiratory infection,” “fever,” and “sore throat” into a trained Word2Vec model or other text-to-vector model, such as vocabulary mapping. The Word2Vec model may be trained to generate a vector representation for each word that represents a symptom. The vector representations for the three symptoms may be averaged to generate a single vector representation for the “symptoms” data element of the target subject record. The single vector representation for the “symptoms” data element of the target subject record may be processed to identify other subject records that include similar words in the “symptoms” data element. Each subject record stored in the database may be associated with an existing “symptoms” data element that has been transformed into a numerical representation, such as a vector. The vector for the “symptoms” data element may be plotted and compared against the vector for the “symptoms” data element of the target subject record. The server may identify the nearest vector to the vector characterizing the “symptoms” data element. The vector of the “symptoms” data element nearest the vector of the target subject record may be predicted to be similar to the subject. The subject record associated with the nearest vector to the vector of the target subject record may be identified and further evaluated to determine the treatment regimen provided to that subject. The treatments that were provided to the subject associated with the vector nearest the vector for the target subject record may be used as potential treatment regimens to treat the target subject. Additionally, each potential treatment regimen may be weighted by the responsiveness experienced by other subject. The potential treatment regimens may be sorted according to the responsiveness that the other subject experienced.

A set of rules may be defined based on a user interaction with a user interface, which may include specifications of particular criteria and an associated particular medical treatment and/or selection of one or more previously defined rules (that specify criteria and a treatment). For example, one or more existing rules may be presented via an interface, and a user may select rules to incorporate into a rule-base associated with an account associated with the user. The one or more rules may be selected from amongst a set of rules defined by multiple users (e.g., associated with one or more institutions) and/or may be generated based on rules generated by multiple users. When a user selects a rule for incorporating into a rule-base, the application may generate a feedback signal to cloud server 135. The feedback signal may include metadata associated with the user's selection. The metadata may indicate whether the rule was incorporated into the rule-base without modification or with modification. If the rule-base was modified, then the metadata would indicate which modification was made to the rule. The metadata may also indicate whether or not the rule was rejected, deleted, or otherwise determined not to be useful to the user. To illustrate and as a non-limiting example, a computing system may detect that rules that relate one or more particular types of symptoms and/or test results to a given treatment are relatively frequently defined and/or selected by users, and the computing system may then generate a general rule pertaining to the particular types of symptoms and/or test results and to the treatment. The general rule may be defined to have (for example) a most restrictive, most inclusive or median criteria. In some instances, a rule base of a user can be processed to detect any criteria overlap between rules. Upon identifying an overlap, an alert may be presented that identifies the overlap. A rule of a rule base may be used to evaluate a subject record to classify to define a population associated with the subject record. Evaluating the subject record using the rule may be performed as a decision tree, for example, in that a first criterion of the rule is compared against the attributes included in the subject record. If the first criterion is satisfied, then the next criterion is compared against the attributes included in the subject record. If the next criterion is satisfied, then the comparisons continue for each criterion included in the rule. The comparisons may continue even if the next criterion is not satisfied. In this case, the non-satisfaction of the criterion (and any others included in the rule) is stored and presented to a user device, along with the criteria that were satisfied.

Accordingly, embodiments of the present disclosure provide a cloud-based application configured to exchange subject information with external entities without violating data-privacy rules. The cloud-based application is configured to automatically assess data-privacy rules involved in sharing subject information across various jurisdictions. The cloud-based application is configured to execute protocols that obfuscate or otherwise modify the subject information, thereby algorithmically ensuring compliance with the data-privacy rules.

IV. Network Environment for Hosting the Cloud-Based Application Configured with Intelligent Functionality

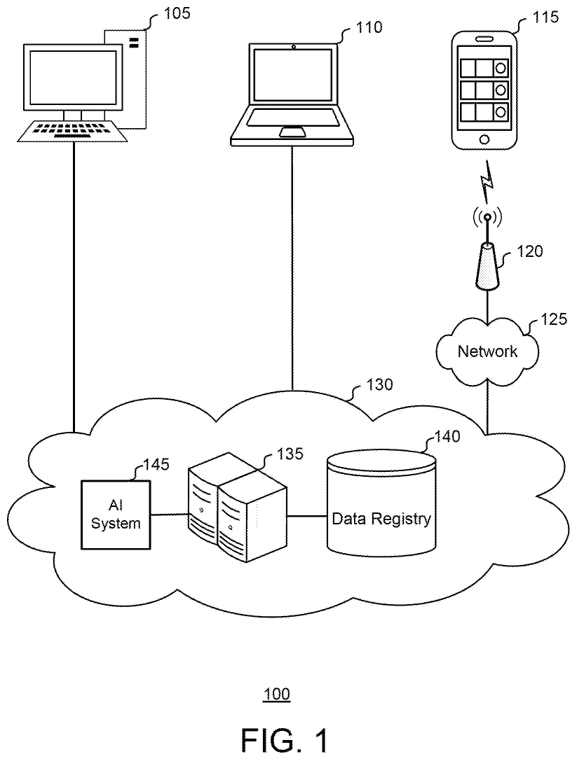

FIG. 1 illustrates network environment 100, in which an embodiment of the cloud-based application is hosted. Network environment 100 may include cloud network 130, which includes cloud server 135, data registry 140, and AI system 145. Cloud server 135 may execute the source code underlying the cloud-based application. Data registry 140 may store the data records ingested from or identified using one or more user devices, such as computer 105, laptop 110, and mobile device 115.

The data records stored in data registry 140 may be structured according to a skeleton structure of fixed parts (e.g., data elements). Computer 105, laptop 110, and mobile device 115 may each be operated by various users. For example, computer 105 may be operated by a physician, laptop 110 may be operated by an administrator of an entity, and mobile device 115 may be operated by a subject. Mobile device 115 may connect to cloud network 130 using gateway 120 and network 125. In some examples, each of computer 105, laptop 110, and mobile device 115 are associated with the same entity (e.g., the same hospital). In other examples, computer 105, laptop 110, and mobile device are associated with different entities (e.g., different hospitals). The user devices of computer 105, laptop 110, and mobile device 115 are examples for the purpose of illustration, and thus, the present disclosure is not limited thereto. Network environment 100 may include any number or configuration of user devices of any device type.

In some embodiments, cloud server 135 may obtain data (e.g., subject records) for storing in data registry 140 by interacting with any of computer 105, laptop 110, or mobile device 115. For example, computer 105 interacts with cloud server 135 by using an interface to select subject records or other data records stored locally (e.g., stored in a network local to computer 105) for ingesting into data registry 140. As another example, computer 105 interacts with an interface to provide cloud server 135 with an address (e.g., a network location) of a database storing subject records or other data records. Cloud server 135 then retrieves the data records from the database and ingests the data records into data registry 140.

In some embodiments, computer 105, laptop 110, and mobile device 115 are associated with different entities (e.g., medical centers). The data records that cloud server 135 obtains from computer 105, laptop 110, and mobile device 115 may be stored in different data registries. While the data records from each of computer 105, laptop 110, and mobile device 115 may be stored within cloud network 130, the data records are not intermingled. For example, computer 105 cannot access the data records obtained from laptop 110 due to the constraints imposed by data-privacy rules. However, cloud server 135 may be configured to automatically obfuscate, obscure, or mask portions of the data records when those data records are queried by a different entity. Thus, the data records ingested from an entity may be exposed to a different entity in an obfuscated, obscured, or masked form to comply with data-privacy rules.

Once the data records are collected from computer 105, laptop 110, and mobile device 115, the data records may be used as training data to train machine-learning or artificial-intelligence models to provide the intelligent analytical functionality described herein. The data records may also be available for querying by any entity, given that when a user device associated with an entity queries data registry 140 and the query results include data records originating from a different entity, those data records may be provided or exposed to the user device in an obfuscated form, which complies with data-privacy rules.

Cloud server 135 may be configured in a specialized manner to execute code that, when executed, causes intelligent functionality to be performed using transformed representations of subject records (e.g., a vector that numerically represent the information stored in a subject record). For example, intelligent functionality may be performed by executing code using cloud server 135. The executed code may represent a trained neural network model. The neural network model may have been trained to perform intelligent functions, such as predicting a subject's responsiveness to a treatment regimen, identifying similar patients, generating a recommendation of a treatment regimen for a patient, and other intelligent functionality. The neural network model may be trained using a training data set that includes subject records of subjects who have previously been treated for a condition and experienced an outcome (e.g., overcoming a condition, increasing a severity of a condition, reducing a severity of a condition, and so on). Additionally, the executed code may be configured to cause cloud server 135 to transform non-numerical values of existing subject records into numerical representations (e.g., a transformed representation), which can be processed by the trained neural network model. For example, the code executed by cloud server 135 can be configured to receive as input each subject record of a set of subject records, and for each subject record, the code, when executed, can cause cloud server 135 to perform the operations described herein for transforming each data element of each subject record into a transformed representation, such as a vector representation. Executing intelligent functionality may include inputting at least a portion of the data records stored in data registry 140 into a trained machine-learning or artificial-intelligence models to generate outputs for further analysis. In some embodiments, the outputs can be used to extract patterns within the data records or to predict values or outcomes associated with data fields of the data records. Various embodiments of the intelligent functionality executed by cloud server 135 are described below.

In some embodiments, cloud server 135 is configured to enable a user device (e.g., operated by a doctor) to access the cloud-based application to transmit consult broadcasts to a set of destination devices. A consult broadcast may be a request for support or assistance regarding the treatment of a subject associated with a subject record. A destination device may be a user device operated by another user associated with another entity (e.g., a doctor at another medical center). If a destination device accepts the request for assistance associated with the consult broadcast, the cloud-based application may generate a condensed representation of the subject record that omits or obscures certain data fields of the subject record. The condensed representation may comply with data-privacy rules, and thus, the condensed representation of the subject record cannot be used to uniquely identify the subject associated by the subject record. The cloud-based application may transmit the condensed representation of the subject record to the destination device that accepted the request for assistance. The user operating the destination device may evaluate the condensed representation and communicate with the user device using a communication channel to discuss options for treating the subject. For example, the communication channel may be configured as a secure chatroom that enables the user device (e.g., operated by the doctor requesting the consult) to securely communicate with the destination device (e.g., operated by the other doctor providing the consult).

In some embodiments, cloud server 135 is configured to provide a treatment-plan definition interface to user devices. The treatment-plan definition interface enables user devices to define a treatment plan for a condition. For example, a treatment plan may be a workflow for treating a subject with the condition. A workflow may include one or more criteria for defining a population of subjects as having the condition. The workflow may also include a particular type of treatment for the condition. The cloud server 135 receives and stores treatment-plan definitions for a particular condition from each user device of a set of user devices. The cloud-based application may distribute a treatment plan for a given condition to a set of user devices. Two or more user devices of the set of user devices may be associated with different entities. Each of the two or more users devices may be provided with the option to integrate any portion or the entire treatment plan into a customer rule set. Cloud server 135 can monitor whether user devices integrate the shared treatment plan in full or integrate part of the treatment plan. The interactions between the user devices and the shared treatment plan can be used to determine whether to update the treatment plan or a rule created based on the treatment plan.

In some embodiments, cloud server 135 enables a user operating a user device to access the cloud-based application to determine a proposed treatment for a subject with a condition. The user device loads an interface associated with the cloud-based application. The interface enables the user operating the user device to select a subject record associated with a subject being treated by the user. The cloud-based application may evaluate other subject records to identify a previously-treated subject who is similar to the subject being treated by the user. The similarity between subjects, for example, may be determined using an array representation of the subject records. An array representation (e.g., a transformed representation, such as a vector, an N-dimensional matrix, or any numerical representation of a non-numerical value) may be any numerical and/or categorical representation of the values of data fields of a subject record. For example, an array representation of a subject record may be a vector representation of the subject record in a domain space, such as in a Euclidean space. In some instances, cloud server 135 may be configured to transform an entire subject record into a numerical representation, such as a vector. For a given subject record, cloud server 135 may evaluate each data element to determine the type of data contained or included in that data element. The type of data may inform the cloud server 135 as to which process or technique to perform to transform the numerical or non-numerical values of that data element into a numerical representation. As an illustrative example, cloud server 135 may transform non-numerical values (e.g., the text of a physician's notes) of a data element of a subject record into a numerical representation (e.g., a vector). The transformation may include using natural language processing techniques, such as Word2Vec or other text vectorization techniques, to generate a numerical value that represents each word of text. The generated numerical value may serve as a vector that can be inputted into a trained neural network to perform intelligent analysis. As another illustrative example, for data elements that include images (e.g., MRI data) or image frames of a video (e.g., a video data of an ultrasound), each image or image frame may be transformed into a numerical representation (e.g., vector) using a trained auto-encoder neural network, which is trained to generate a latent-space representation of an input image. The condensed representation of the input image (e.g., the latent-space representation) may serve as the numerical representation of the input image. This numerical representation can be inputted into a neural network or other machine-learning model to perform intelligent analysis of the associated subject record. As yet another example, for data elements that include a time-variant sequence of information (e.g., events occurring or measurements taken from a subject over a period of time), the time-variant information can be represented as a numerical representation using several exemplary transformations. In some instances, the count of events may be used as the vector representing the time-variant information. For example, if a measurement was taken with respect to a subject four times in one year, the numerical representation may be “4.” In other instances, the frequency or rate of events occurring (e.g., per week, per month, per year, etc.) may be used as the vector representing the time-variant information. In still other instances, an average or combination of the measurement values associated with each event in the time-variant information can be used as the vector representing the time-variant information. The present disclosure is not limited to these examples, and thus, other numerical representations of time-variant information can be used as the vector that represents the numerical representation.

AI system 145 can be configured to collect data sets at a big-data scale, transform the collected data sets into curated training data, execute learning algorithms using the curated training data, and storing the detected patterns, correlations, and/or relationships of the training data in one or more trained AI models. In some implementations, AI system 145 can be configured to perform certain predictive functionality, such as, predicting a disease progression for a particular subject with SMA, predicting candidate subject groups for including in new or existing clinical studies, or predicting a contextual treatment schedule specific to the particular subject. In some implementations, as described in greater detail with respect to FIGS. 8 and 11, the output of AI system 145 can be predictive of the disease progression for a particular subject diagnosed with SMA. In other implementations, as described in greater detail with respect to FIGS. 9 and 12, the output of AI system 145 can be predictive of new groupings of subjects who may be suitable candidates for a new clinical study. In other implementations, as described in greater detail with respect to FIGS. 10 and 13, the output of AI system 145 can be predictive of a treatment selection for a particular subject with SMA.

In some instances, multiple values in an array representation correspond to a single field. For example, a value of a data element may be represented by multiple binary values generated via one-hot encoding. As another example, each value of the multiple values in a single data element of a subject record may be individually transformed into a numerical representation, as described above. The numerical representation that represents each value of the multiple values can be combined into a single numerical representation that corresponds to the data element. Combining multiple numerical representations may be performed using any vector combination techniques, such as averaging vector magnitudes, adding vectors, or concatenating multiple vectors into a single vector. In some instances, the cloud-based application may generate array representations for each subject record of a group of subject records. Similarity between two subject records may be represented by comparing the two array representations to determine a distance between them. Subject records can also be compared along a dimension (e.g., a data element), instead of comparing a numerical representation of an entire subject record with another numerical representation of another subject record. For example, comparing two subject records along a dimension may include comparing the numerical representation of a data element of a subject record with another numerical representing of a matching data element of another subject record. Further, the cloud-based application may be configured to identify a subject who is a nearest neighbor to the subject record selected by the user device using the interface. The nearest neighbor may be determined by comparing the numerical representations of the various subject records with the numerical representation of a target subject record. The cloud-based application may identify treatments previously performed on the subject who is the nearest neighbor. The cloud-based application may avail on the interface the previously-performed treatments on the nearest neighbor.

In some embodiments, cloud server 135 is configured to create queries that search a database of previously-treated subjects. Cloud server 135 may execute the queries and retrieve subject records that satisfy the constraints of the query. In presenting the query results, however, the cloud-based application may only present the subject record in full for subjects who have been or who are being treated by the user who created the query. The cloud-based application masks or otherwise obfuscates portions of subject records for subjects who are not being treated by the user creating the query. The masking or obfuscation of portions of subject records that are included in the query results enables the user to comply with data-privacy rules. In some embodiments, the query results (regardless of whether the query results are obfuscated or not) can be automatically evaluated for patterns or common attributes within the subject records.

In some embodiments, cloud server 135 embeds a chatbot into the cloud-based application. The chatbot is configured to automatically communicate with user devices. The chatbot can communicate with a user device in a communication session, in which messages are exchanged between the user device and the chatbot. A chatbot may be configured to select answers to questions received from user devices. The chatbot may select answers from a knowledge base accessible to the cloud-based application. When a user device transmits a question to the chatbot, and that chatbot does not have a preexisting answer stored in the knowledge base, then a different representation of the question for which there is a preexisting answer stored in the knowledge base. The user communicating with the chatbot can be prompted as to whether the answer provided by the chatbot is accurate or helpful.

It will be appreciated that any machine-learning or artificial-intelligence algorithms may be executed to generate any of the trained machine-learning models described herein. Various different types and technologies of artificial-intelligence-based and machine-learning models may be trained and then executed to generate one or more outputs predictive of user outcomes for performing a protocol or function. Non-limiting examples of models include Naive Bayes models, random forest or gradient boosting models, logistic regression models, deep learning neural networks, ensemble models, supervised learning models, unsupervised learning models, collaborative filtering models, and any other suitable machine-learning or artificial intelligence models.

It will be appreciated that the cloud-based application can be configured to perform intelligent functionality with respect to consulting external physicians, determining diagnosis and proposing treatment for any disease, condition, area of study, or disorder, including, but not limited to, COVID-19, oncology, including cancers of the lung, breast, colorectal, prostate, stomach, liver, cervix uteri (cervical), esophagus, bladder, kidney, pancreas, endometrium, oral, thyroid, brain, ovary, skin, and gall bladder; solid tumors, such as sarcomas and carcinomas, cancers of the immune system including lymphomas (such as Hodgkin or non-Hodgkin), and cancers of the blood (hematological cancers) and bone marrow, such as leukemias (such as Acute lymphocytic leukemia (ALL) and Acute myeloid leukemia (AML)), lymphomas, and myeloma. Additional disorders include blood disorders such as anemia, bleeding disorders such as hemophilia, blood clots, ophthalmology disorders, including diabetic retinopathy, glaucoma, and macular degeneration, neurological disorders, including multiple sclerosis, Parkinson's, disease, spinal muscular atrophy, Huntington's Disease, amyotrophic lateral sclerosis (ALS), and Alzheimer's Disease, autoimmune disorders, including multiple sclerosis, diabetes, systemic lupus erythematosus, myasthenia gravis, inflammatory bowel disease (IBD), psoriasis, Guillain-Barre syndrome, Chronic inflammatory demyelinating polyneuropathy (CIDP), Graves' disease, Hashimoto's thyroiditis, eczema, vasculitis, allergies and asthma.

Other diseases and disorders include but are not limited to kidney disease, liver disease, heart disease, strokes, gastrointestinal disorders such as celiac disease, Crohn's disease, diverticular disease, Irritable Bowel Syndrome (IBS), Gastroesophageal Reflux Disease (GERD) and peptic ulcer, arthritis, sexually transmitted diseases, high blood pressure, bacterial and viral infections, parasitic infections, connective tissue diseases, celiac disease, osteoporosis, diabetes, lupus, diseases of the central and peripheral nervous systems, such as Attention deficit/hyperactivity disorder (ADHD), catalepsy, encephalitis, epilepsy and seizures, peripheral neuropathy, meningitis, migraine, myelopathy, autism, bipolar disorder, and depression.

IV.A. The Cloud-Based Application Enables User Devices to Broadcast Consult Requests to Other User Devices and Automatically Condenses Subject Records to Comply with Data-Privacy Rules

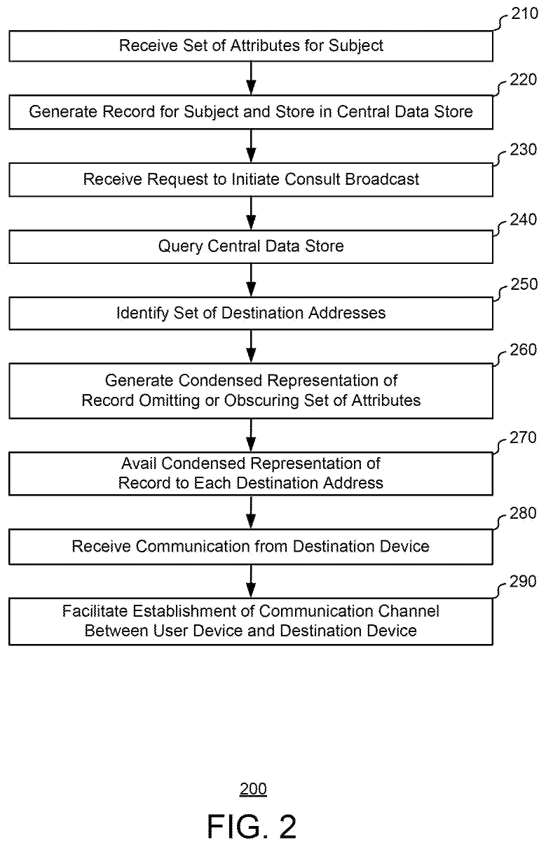

FIG. 2 is a flowchart illustrating process 200 performed by the cloud-based application to distribute condensed subject records to user devices in association with a consult broadcast requesting assistance with treating a subject. Process 200 may be performed by cloud server 135 to enable user devices associated with different entities (e.g., hospitals) to collaborate or consult regarding treatment for a subject, while complying with data-privacy rules.

Process 200 begins at block 210 where cloud server 135 receives a set of attributes from a user device. Each attribute of the set of attributes can represent any characteristic(s) of a subject (e.g., a patient). The set of attributes may be identified by a user using an interface provided by cloud server 135. For example, the set of attributes identify demographic information of the subject and a recent symptom experienced by the subject. Non-limiting examples of demographic information include age, sex, ethnicity, state or city of residence, income range, education level, or any other suitable information. Non-limiting examples of a recent symptom include a subject currently or recently (e.g., at a last visit, at intake, within 24 hours, within a week) experienced a particular symptom (e.g., difficulty breathing, fever above a threshold temperature, blood pressures above a threshold blood pressure, etc.).

At block 220, cloud server 135 generates a record for the subject. The record may be a data element including one or more data fields. The record indicates each of the set of attributes associated with the subject. The record may be stored at a central data store, such as data registry 140 or any other cloud-based database. At block 230, cloud server 135 receives a request, which was submitted by a user using the interface. The request may be to initiate a consult broadcast. For example, the user associated with an entity is a physician at a medical center treating a subject. The user can operate a user device to access the cloud-based application to broadcast a request for assistance with treating the subject. The broadcast may be transmitted to a set of other user devices associated with a different entity.

At block 240, cloud server 135 queries the central data store using the one or more recent symptoms included in the set of attributes associated with a subject. The query results include a set of other records. Each record of the set of other records is associated with another subject. In some instances, cloud server 135 may query the central data store to identify other subject records that are similar to the subject record. Similarity may be determined by comparing the transformed representation of the entire subject record to the transformed representation of each other subject record. The comparison of the transformed representations may result in a distance (e.g., a Euclidean distance) that represents a degree of similarity between the two subject records. In other instances, similarity may be determined based on values included in a data element. For example, a target subject record may include a target data element including text that represents symptoms experienced by a subject. Each other subject record stored in the central data store may also include a data element including text that represents the symptoms of the associated subject. Cloud server 135 can transform the text included in the target data element into a numerical representation using techniques described above (e.g., a trained convolution neural network, a text vectorization technique, such as Word2Vec, etc.). The numerical representation of the text included in the target data element may be compared against the numerical representation of the text included in the matching data element of each other subject record. The result of the comparison (e.g., in a domain space, such as a Euclidean space) between two numerical representations may indicate a degree to which the text included in the target data element is similar to the text included in the data element of another subject record. At block 250, cloud server 135 identifies a set of destination addresses (e.g., other user devices associated with a different entity). Each destination address of the set of destination address is associated with a care provider for another subject associated with one or more other records of the set of other records identified at block 240. At block 260, cloud server 135 generates a condensed representation of the record for the subject. The condensed representation of the record omits, obscures, or obfuscates at least a portion of the record. The condensed representation of the record can be exchanged between external systems without violating data-privacy rules because the condensed representation of the record cannot be used to uniquely identify the subject associated with the record. Cloud server 135 can execute any masking or obfuscation techniques to generate the condensed representation of the record.

At block 270, cloud server 135 avails the condensed representation of the record with a connection input component (e.g., a selectable link, such as a hyperlink, that causes a communication channel to be established) to each destination address of the set of destination addresses. The connection input component may be a selectable element presented to each destination address. Non-limiting examples of the connection input component include a button, a link, an input element, and other suitable selectable elements. At block 280, cloud server 135 receives a communication from a destination device associated with a destination address. The communication includes an indication that the user operating the destination device selected the connection input component associated with the condensed representation of the record. At block 290, cloud server 135 establishes a communication channel between the user device and the destination device at which the connection input component was selected. The communication channel enables the user operating the user device (e.g., the physician treating the subject) to exchange messages or other data (e.g., a video feed) with the destination device associated with the destination address at which the connection input component was selected (e.g., a physician at another hospital who agreed to assist with the treatment of the patient).

In some embodiments, cloud server 135 is configured to automatically determine a location of the user device and a location of the destination device at which the connection input component was selected. Cloud server 135 can also compare the locations to determine whether to generate the condensed representation of the record. For example, at block 260, cloud server 135 may generate the condensed representation of the record because cloud server 135 determines that each destination address of the set of destination addresses is not collocated with the user device that initiated the consult broadcast. In this case, cloud server 135 may automatically determine to generate the condensed representation of the record to comply with data-privacy rules. As another example, if the set of destination addresses is associated with the same entity as the user device that initiated the consult broadcast, then cloud server 135 can transmit the record in full (e.g., without obfuscating a portion of the record) to a destination device associated with a destination address, while still complying with the data-privacy rules.

In some embodiments, cloud server 135 generates a plurality of other condensed record representations. Each of the plurality of other condensed record representations is associated with another subject. Cloud server 135 transmits the plurality of other condensed record representations to the user device; and receives, from the user device, a communication identifying selections of a subset of the plurality of other condensed record representations. Each of the set of destination addresses is represented by one of the condensed record representations. For example, generating a condensed record representation includes determining a jurisdiction of another subject associated with the condensed record representation, determining a data-privacy rule governing the exchange of subject records within the jurisdiction, and generated the condensed record representation to comply with the data-privacy rule. A first other condensed record representation of the plurality of other condensed record representations may include data of a particular type. A second other condensed record representation of the plurality of other condensed record representations may omit or obscure data of the particular type. For example, data of the particular type may be contact information, identifying information, such as name, social security number, and other suitable information that can be used to uniquely identify the other subject.

In some implementations, a communication may be received at the central data store. The communication may be transmitted by a user device operated by a user and may include an identifier of a target subject record of a target subject. The communication, when received at the central data store, may cause the central data store to query the stored set of subject records to identify an incomplete subset of the set of subject records. Each subject record of the incomplete subset may be identified and included in the incomplete subset because the subject record is determined to be similar to the target subject record along at least one dimension. Similarity between two subject records along a dimension may represent similarity with respect to a data element of the subject records, such as similarity with respect to symptoms, diagnoses, treatments, or any other suitable data elements. The one or more dimensions, along which similarity or dissimilarity is determined, may be defined automatically or may be user defined. Determining a similarity or dissimilarity between the target subject record and each subject record of the set of subject records stored in the central data store may include at least the following operations: retrieving the target subject record based on the identifier included in the communication, generating a transformed representation of the target subject record (or retrieving the existing transformed representation of the target subject record), and performing a clustering operation using the transformed representation of the target subject record and the transformed representation of each subject record of the set of subject records. The clustering operation may be performed with respect to one or more dimensions (e.g., one or more features of a subject record). For example, the clustering operation may cluster the set of subject records stored in the central data store based on the data element that contains values representing a subject's symptoms. The transformed representation of the target subject record may include a vector representation of the data element that contains values representing the subject's symptoms. The vector representation of this data element of the target subject record and the vector representations of the corresponding data element in each subject record of the set of subject records may be compared to define clusters of subject records. Each cluster of subject records may define a group of one or more subject records that share a common characteristic associated with the data element selected as the dimension of similarity. In each cluster of subject records, a Euclidean distance may be computed between the transformed representation of the target subject record and the other transformed representations of the set of subject records. A subject record may be determined to be similar to the target subject record when, for example, the Euclidean distance between the transformed representation of the subject record and the transformed representation of the target subject record is within a threshold value.

IV.B. Updating Shareable Treatment-Plan Definitions Based on Aggregated User Integration

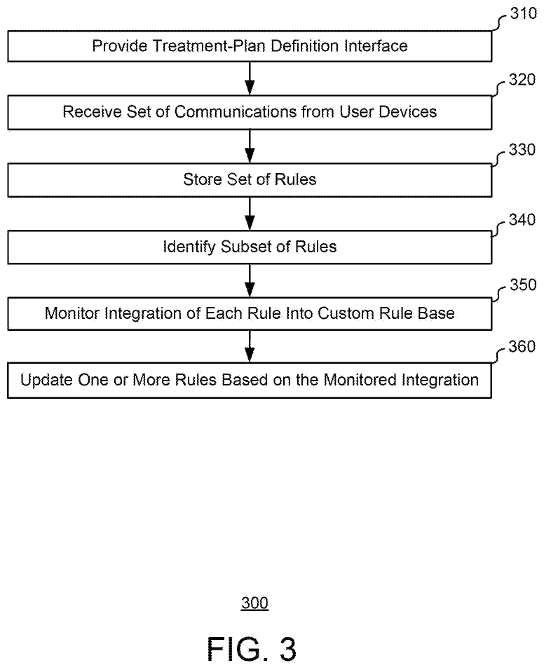

FIG. 3 is a flowchart illustrating process 300 for monitoring the user integration of treatment-plan definitions (e.g., decision trees or treatment workflows) and automatically updating the treatment-plan definitions based on a result of the monitoring. Process 300 may be performed by cloud server 135 to enable a user device to define a treatment plan for treating a population of subjects with a condition. The user device may distribute the treatment-plan definition to user devices connected to internal or external networks. The user devices receiving the treatment-plan definition can determine whether to integrate the treatment-plan definition into a custom rule base. The integration into the custom rule base can be monitored and used to automatically modify the treatment-plan definition.

At block 310, cloud server 135 stores interface data that causes a treatment-plan definition interface to be displayed when a user device loads the interface data. The treatment-plan definition interface is provided to each user device of a set of user devices when the user devices accesses cloud server 135 to navigate to the treatment-plan definition interface. In some embodiments, the treatment-plan definition interface enables a user to define a treatment plan for treating a population of subjects that have a condition (e.g., lymphoma).

At block 320, cloud server 135 receives a set of communications. Each communication of the set of communications is received from a user device of the set of user devices and was generated in response to an interaction between the user device and the treatment-plan definition interface. In some embodiments, the communication includes one or more criteria, for example, for defining a population of subject records. Each criteria may be represented by a variable type. For example, variable type may be a value or variable used as the condition of a criteria. The variable type of a criterion of a rule may also be any value of a condition that constrains the population of subjects to an incomplete sub-group. For example, the variable type of a rule that defines a population of pregnant women is “IF ‘subject is pregnant.’” A criterion may be a filter condition for filtering a pool of subject records. For example, a criteria for defining a population of subject records associated with subjects who may develop a lymphoma may include a filter condition of “abnormality in anaplastic lymphoma kinase (ALK)” AND “over 60 years old.” The communication may also include a particular type of treatment for the condition. The particular type of treatment may be associated with a certain action (e.g., undergo surgery) or refraining from certain action (e.g., reduce salt intake) that is proposed to treat the condition associated with the subjects represented by the population of subject records.

At block 330, cloud server 135 stores a set of rules in a central data store, such as data registry 140 or any other centralized server within cloud network 130. Each rule of the set of rules includes the one or more criteria and the particular treatment type included in the communication from a user device. As an illustrative example, a rule represents a treatment workflow for treating lymphoma in a subject. The rule includes the following criteria (e.g., the conditions following the “IF” statement) and a next action (e.g., the particular treatment type defined or selected by the user, and which follow the “THEN” statement): “IF ‘biopsy of lymph nodes indicates lymphoma cells are present’ AND ‘blood test reveals lymphoma cells present’ THEN ‘treat with chemotherapy’ AND ‘active surveillance.’” Additionally, each rule of the set of rules is stored in association with an identifier corresponding to the user device from which the communication was received.

At block 340, cloud server 135 identifies a subset of the set of rules that are available across entities via the treatment-plan definition interface. A subset of rules may include the subset of the set of rules associated with a condition and that are distributed to external systems, such as other medical centers, for evaluation. For example, a rule can be selected for including in the subset of rules by evaluating a characteristic of the rule or the identifier associated with the rule. The characteristic of the rule can include a code or flag stored or appended to the stored rule. The code or flag indicates the rule is generally available to external systems (e.g., availed to entities).

At block 350, for each rule of the subset of rules identified at block 340, cloud server 135 monitors interactions with the rule. An interaction may include an external entity (e.g., external to the entity associated with the user who defined the treatment plan associated with the rule) integrating the rule into a custom rule base. For example, a user device associated with an external entity (e.g., a different hospital) evaluates the rule availed to the external entity. The evaluation includes determining whether the rule is suitable for integrating into a rule set defined by the external entity. The rule may be suitable when the user device associated with the external entity indicates that the treatment workflow that is defined using the rule is suitable to treat the condition corresponding to the rule. Continuing with the illustrative example above, the rule for treating lymphoma may be availed to an external medical center. A user associated with the external medical center determines that the rule for treating lymphoma is suitable for integrating into the rule set defined by the external medical center. Thus, after the rule is integrated into a custom rule base defined by the external medical center, other users associated with the external medical center will be able to execute the integrated rule by selecting the integrated rule from the custom rule base. Additionally, cloud server 135 monitors integration of the availed rule by detecting a signal generated or caused to be generated when the treatment-plan definition interface receives input corresponding to an integration of the rule into the custom rule base from the user device associated with the external entity.

As another illustrative example, the user device associated with the external entity uses the treatment-plan definition to integrate an interaction-specified modified version of the rule into the custom rule base. The interaction-specified modified version of the rule is a portion of the rule selected for integration into the custom rule base. Selecting a portion of the rule for integration includes selecting less than all criteria included in the rule for integration into the custom rule base. Continuing with the illustrative example above, the user device associated with the external entity selects the criteria of “IF ‘biopsy of lymph nodes indicates lymphoma cells are present’” for integration into the custom rule base, but the user device does not select the criteria of “blood test reveals lymphoma cells present” for integration into the custom rule base. Thus, the interaction-specific modified version of the rule integrated into the custom rule base is “IF ‘biopsy of lymph nodes indicates lymphoma cells are present’” THEN ‘treat with chemotherapy’ AND ‘active surveillance.’” The criteria of “blood test reveals lymphoma cells present” is removed from the rule to create the interaction-specified modified version of the rule, which is integrated into the custom rule base.

At block 360, cloud server 135 may detect that the interaction-specified modified version of the rule was integrated into the custom rule base defined by the external entity. Once detected, cloud server 135 may update the rule stored at the central data store of cloud network 130. The rule may be updated based on the monitored interaction(s). The term “based on” in this example corresponds to “after evaluating” or “using a result of an evaluation of” the monitored interaction(s). For example, cloud server 135 detects that the user device associated with the external entity integrated the interaction-specified modified version of the rule. In response to detecting the interaction-specified modified version of the rule, cloud server 135 may update the rule stored in the central data store from the existing rule to the interaction-specified modified version of the rule.

In some embodiments, cloud server 135 updates the rule by generating an updated version that is to be availed across external entities. Another original version may remain un-updated and is availed to a user associated with the user device from which the one or more communications that identified the criteria and particular type of treatment was received. For example, cloud server 135 updates the rule stored at the central data store, but cloud server 135 does not update another rule of the set of rules stored at the central data store.

In some embodiments, cloud server 135 may update the rule when an update condition has been satisfied. An update condition may be a threshold value. For example, the threshold value may be a number or percentage of external entities that have integrated a modified version of the rule into their custom rule bases. As another example, the update condition may be determined using an output of a trained machine-learning model. To illustrate, cloud server 135 may input the detected signals received from external entities into a multi-armed bandit model that automatically determines whether and/or when to avail the rule and/or whether and when to avail an updated version of the rule. To illustrate and only as a non-limiting example, a rule may be defined as executable code, such that the rule, upon execution, automatically queries the central data store to identify a subset of the set of subject records to further analyze. Additionally, the rule may include one or more treatment protocols for treating the subjects associated with the identified subset of subject records. The rule may be defined as a workflow for defining a subset of the set of subject records and treating the subset associated with the subset of subject records. For example, the rule may include one or more criteria for filtering subject records out of the set of subject records, and for performing certain treatment protocols on the subjects associated with the remaining subject records (e.g., the subject records remaining after the filtering has been performed on the set of subject records). While the rule is defined by a user of a first entity, the rule may be accepted (e.g., integrated into a rule base of the second entity), modified, or entirely rejected by an external user (e.g., a doctor who works at a different hospital) of a second entity (e.g., the first and second entities being two different medical facilities). In some examples, each time an external user of the second entity accepts the rule, and thus, fully integrates the rule into its codebase, then a feedback signal may be transmitted to the cloud server 135. In other examples, each time a user of the second entity modifies the rule, then a feedback signal may be transmitted to the cloud server 135. In other examples, each time a user of the second entity entirely rejects the rule, then a feedback signal may be transmitted to the cloud server 135. In each example above, the feedback signal may include data indicating the rule (e.g., a rule identifier) and whether the rule was accepted, modified, or rejected. A multi-armed bandit model (executable by cloud server 135) can be configured to intelligently select one of the original rule, the modified rule, or an entirely different rule for broadcasting to external users of other entities. The selection of the original rule, the modified rule, or the different rule may be based at least in part on the configuration of the multi-armed bandit. In some examples, the multi-armed bandit may be configured with an epsilon greedy search technique. In an epsilon greedy search technique, the multi-armed bandit model may select the original rule for broadcasting to external users of other entities with a probability of “1—epsilon,” where epsilon represents a probability of exploring a new or modified rule. Thus, the multi-armed bandit model may select a modified version of the original rule or a completely new rule with a probability of the defined epsilon. The multi-armed bandit model may change the epsilon based on the feedback signals received from the other entities. For example, if the feedback signals indicate that the rule has been modified in a specific manner by different external users over a threshold number of times, then the multi-armed bandit model may learn to select the rule, as modified in the specific manner, to broadcast to external users, instead of broadcasting the original rule.

In some embodiments, cloud server 135 identifies multiple rules of the set of rules that include criteria corresponding to the same variable type and that identify same or similar types of treatment. A variable type may be a value or variable used as the condition of a criteria. The variable type of a criterion of a rule may also be any value of a condition that constrains the population of subjects to a sub-group. For example, the variable type of a rule that defines a population of pregnant women is “IF ‘subject is pregnant.’” Cloud server 135 determines a new rule that is a condensed representation of the multiple rules, when the new rule is generally transmitted to the servers operated by other entities.

In some embodiments, cloud server 135 provides another interface configured to receive a set of attributes of a subject. For example, a user operating a user device to access the other interface and select a subject record that includes a set of attributes using the other interface. The selection of the subject record may cause the cloud server 135 to receive the set of attributes of the subject. Cloud server 135 identifies (e.g., determines) a particular rule for which the criteria are satisfied based on the set of attributes of the subject. For example, the evaluates the set of attributes of the subject record against the criteria of the rules stored in the central data store. To illustrate, if the set of attributes includes a data field containing the value “pregnant,” and if a rule includes a single criteria of “IF ‘subject is pregnant,” then cloud server 135 identifies this rule. Cloud server 135 updates the other interface to present the particular rule and each particular type of treatment associated with the particular rule.

In some embodiments, a criterion of a rule is a variable type that relates to a particular demographic variable and/or a particular symptom-type variable. Non-limiting examples of a demographic variable include any item of information that characterizes a demographic of the subject, such as age, sex, ethnicity, race, income level, education level, location, and other suitable items of demographic information. Non-limiting examples of a symptom-type variable indicate whether a subject currently or recently (e.g., at a last visit, at intake, within 24 hours, within a week) experienced a particular symptom (e.g., difficulty breathing, fainting, fever above a threshold temperature, blood pressures above a threshold blood pressure, etc.).

In some embodiments, cloud server 135 monitors data in a registry of subject records, such as the subject records stored in data registry 140. Cloud server 135 monitors the data in the registry of subject records for each rule of the subset of rules (identified at block 340). Cloud server 135 identifies a set of subjects for which the criteria of the rule were satisfied, and for which the particular treatment was previously prescribed to the subject. Cloud server 135 identifies, for each of the set of subjects, a reported state of the subject as indicated from or using assessment or testing. For example, the reported state is any information characterizing a state of the subject in an aspect, such as whether the subject has been discharged, whether the subject is alive, measurements of the subject's blood pressure, the number of times the subject wakes up during a sleep stage, and other suitable states. Cloud server 135 determines an estimated responsiveness metric of the set of subjects to the particular treatment based on the reported states. For example, if the particular treatment of a rule is to prescribe a medication, the estimated responsiveness metric is a representation of the extent to which the medication addressed a symptom or condition experienced by the subject. As a non-limiting example, the estimated responsiveness metric of the set of subjects may be an average, weighted average, or any summation of a score assigned to each subject of the set of subjects. The score can represent or measure the effectiveness of the subject's responsiveness to the treatment. In some instances, cloud server 135 may generate the score that represents the effectiveness of the subject's responsiveness to the treatment by using a clustering technique. To illustrate and as only a non-limiting example, a set of subject records may represent subjects who previously underwent a particular treatment protocol for treating a condition. Each subject record of the set of subject record may be labeled (e.g., by a user) as having one of a positive responsiveness to the particular treatment protocol, a neutral responsiveness to the particular treatment protocol, or a negative responsiveness to the particular treatment protocol. The set of subject records may then be divided into three subsets (e.g., clusters); a first subset of subject records may correspond to subjects who had a positive responsiveness to the particular treatment protocol, a second subset of subject records may correspond to subjects who had a neutral responsiveness to the particular treatment protocol, and a third subset of subject records may correspond to subjects who had a neutral responsiveness to the particular treatment protocol. Cloud server 135 may transform each subject record of the first subset of subject records into a transformed representation, according to implementations described above. Cloud server 135 may also transform each subject record of the second subset of subject records into a transformed representation, using techniques described above. Lastly, cloud server 135 may transform each subject record of the third subject of subject records into a transformed representation, using the techniques described above. In some implementations, determining a predicted responsiveness of a new subject to the particular treatment protocol may include transforming the new subject record of the new subject into a new transformed representation. The new transformed representation may be compared in a domain space (e.g., a Euclidean space) with the transformed representations of each cluster or subset of subject records. If the new transformed representation is closest to a centroid of the transformed representations associated with the first subset, then the new subject is predicted to have a positive responsiveness to the particular treatment. If the new transformed representation is closest to a centroid of the transformed representations of the second subset, then the new subject is predicted to have a neutral responsiveness to the particular treatment. Lastly, if the new transformed representation is closest to a centroid of the transformed representations of the third subset, then the new subject is predicted to have a negative responsiveness to the particular treatment protocol. A centroid may be a multidimensional average of the transformed representations associated with a subset. Cloud server 135 can cause the subset of the set of rules and the estimated responsiveness metrics of the set of subjects to be displayed or otherwise presented in the treatment-plan definition interface.

IV.C. Presenting Treatment Recommendations with Associated Efficacy Using Treatments Prescribed to Similar Subjects

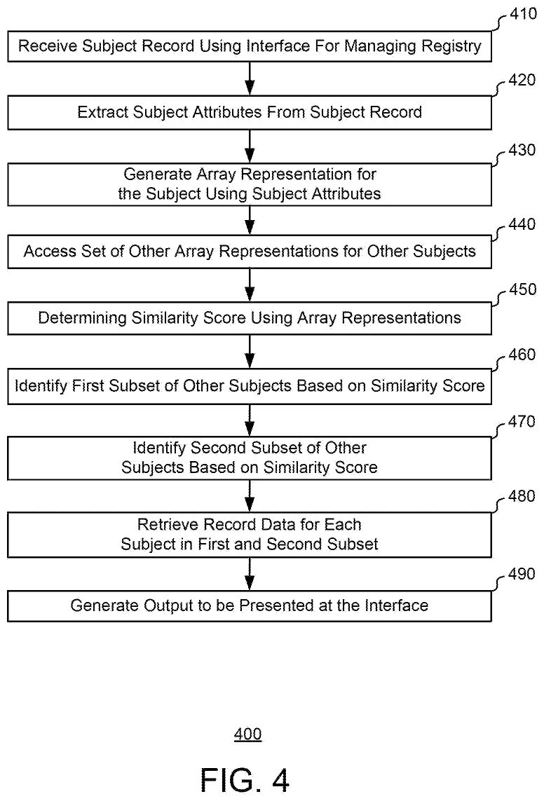

FIG. 4 is a flowchart illustrating process 400 for recommending treatments for a subject. Process 400 can be performed by cloud server 135 to display to a user device associated with a medical entity recommended treatments for a subject and the efficacy of each recommended treatment. The recommended treatments can be identified using a result of evaluating efficacies of treatments previously prescribed to similar subjects.

At block 410, cloud server 135 receives input corresponding to a subject record that characterizes aspects of a subject. The input is received from a user device associated with an entity. Further, the input is received in response to the user device selecting or otherwise identifying the subject record using an interface associated with an instance of a platform configured to manage a registry of subject records. User devices may access the interface by loading interface data stored at a web server (not shown) connected within cloud network 130. The web server may be included or executed on cloud server 135.

At block 420, cloud server 135 extracts a set of subject attributes from the subject record received at block 410. A subject attribute characterizes an aspect of the subject. Non-limiting examples of subject attributes include any information found in an electronic health record, any demographic information, an age, a sex, an ethnicity, a recent or historical symptom, a condition, a severity of the condition, and any other suitable information that characterizes the subject.

At block 430, cloud server 135 generates an array representation of the subject record using the set of subject attributes. For example, the array representation is a vector representation of the values included in the subject record. The vector representation may be a vector in a domain space, such as a Euclidean space. The array representation, however, can be any numerical representation of a value of a data field of the subject record. In some embodiments, cloud server 135 can perform feature decomposition techniques, such as singular value decomposition (SVD), to generate the values representing the set of subject attributes of the array representation of the subject record.

At block 440, cloud server 135 accesses a set of other array representations characterizing multiple other subjects. An array representation included in the set of other array representations may be a vector representation of a subject record that characterizes another subject (e.g., one of the multiple other subjects).

At block 450, cloud server 135 determines a similarity score representing a similarity between the array representation representing the subject and the array representation of each of the other subjects. For example, the similarity score is calculated using a function of a distance (in the domain space) between the array representation representing the subject and the array representation representing the other subject. To illustrate and as only a non-limiting example, the similarity score may be calculated using a range of “0” to “1,” with “0” representing a distance beyond a defined threshold and “1” representing that the array representations have no distance between them. To illustrate and only as a non-limiting example, the similarity score may be based on the Euclidean distance between two array representations (e.g., vectors).

At block 460, cloud server 135 identifies a first subset of the multiple other subjects. Subjects may be included in the first subset when the similarity score associated with a subject is within a predetermined absolute or relative range. Similarly, at block 470, cloud server identifies a second subset of the multiple other subjects. However, subjects may be included in the second subset when the similarity score of this subject is within another predetermine range.

At block 480, cloud server 135 retrieves record data for each subject in the first subset and in the second subset of the multiple other subjects. The record data include the attributes that are included in a subject record characterizing a subject. For example, the subject record data identifies a treatment received by the subject and the subject's responsiveness to the treatment. The responsiveness to the treatment may be represented by text (e.g., “subject responded positively to treatment”) or a score indicating an extent to which the subject responded positively or negatively to the treatment (e.g., a score from “0” to “1” with “0” indicating a negative responsiveness and “1” indicating a positive responsiveness). In some instances, a treatment responsiveness may indicate a degree to which a subject responded positively to a treatment that was previously performed on the subject. For example, the treatment responsiveness may be a numerical (e.g., a score from “0” to “10”) or non-numerical value (e.g., a word assigned to represent the responsiveness, such as “positive,” “neutral,” or “negative”). In some examples, the treatment responsiveness for previously treated subjects may be user defined. In other examples, the treatment responsiveness may be determined automatically based on a result of a test or a measurement taken from the user. For example, the treatment responsiveness may be determined automatically based on values included in a blood test performed on the subject.

At block 490, cloud server 135 generates an output to be presented at the interface on the user device. The output may indicate, for example, a recommendation of one or more treatments for the subject. The recommendation of one or more treatments may be determined based on, for example, the treatments received by the other subjects in the first and second subsets, the treatment responsiveness of subjects in the first and second subsets, and the differences between the subject attributes of subjects in the second subset and subject attributes of the subject.

In some embodiments, cloud server 135 determines that the subject and one of the subjects from the first or second subset are being treated or were treated by the same medical entities. Cloud server 135 determines that the subject and another subject of the first or second subset are being treated or were treated by different medical entities. Cloud server 135 may avail differentially obfuscated versions of records of the subjects via the interface. The cloud-based application can automatically provide differently obfuscated versions of records to entities based on varying constraints imposed on data sharing by the data-privacy rules of different jurisdictions. In some embodiments, cloud server 135 identifies the first subset and the second subset of subject records by performing a clustering operation on the transformed representations of a set of subject records.

IV.D. Automatically Obfuscating Query Results From External Entities

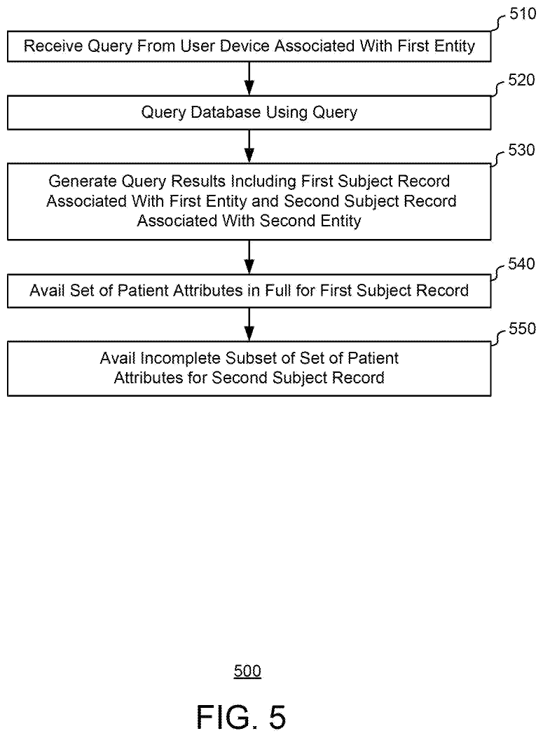

FIG. 5 is a flowchart illustrating process 500 for obfuscating query results to comply with data-privacy rules. Process 500 may be performed by cloud server 135 as an executing rule that ensures data sharing of subject records with external entities complies with data-privacy rules. The cloud-based application may enable a user device to query data registry 140 for subject records that satisfy a query constraint. The query results, however, may include data records originating from external entities. Thus, process 500 enables cloud server 135 to provide user devices with additional information on treatments from external entities, while complying with data-privacy rules.

At block 510, cloud server 135 receives a query from a user device associated with a first entity. For example, the first entity is a medical center associated with a first set of subject records. The query may include a set of symptoms associated with a medical condition or any other information constraining a query search of data registry 140.

At block 520, cloud server 135 queries a database using the query received from the user device. At block 530, cloud server 135 generates a data set of query results that correspond to the set of symptoms and are associated with the medical conditions. For example, the user device transmits a query for subject records of subjects who have been diagnosed with lymphoma. The query results include at least one subject record from the first set of subject records (which originate or were created at the first entity) and at least one subject record from a second set of subject records associated with a second entity (e.g., a medical center different from the first entity). Each of the subject record from the first set of subject records and the subject record from the second set of subject records may include a set of subject attributes. A subject attribute can characterize any aspect of a subject.

At block 540, cloud server 135 presents (e.g., avails or otherwise makes available) to the user device the set of subject attributes in full for subject records included in the first set of subject records because these records originate from the first entity. Presenting a subject record in full includes making the set of attributes included in a subject record available to the user device for evaluation or interaction using the interface. At block 550, cloud server 135 also or alternatively avails to the user device an incomplete subset of the set of subject attributes for each subject record included in the second set of subject records. Providing an incomplete subset of the set of subject attribute provides anonymity to subjects because the incomplete subset of subject attributes cannot be used to uniquely identify a subject. For example, providing an incomplete subset may include available four of 10 subject attributes to anonymize the subject associated with the 10 subject attributes. In some embodiments, at block 550, cloud server 135 avails an obfuscated set of subject attributes for each subject record included in the second subject. Obfuscating the set of attributes include reducing the granularity of information provided. For example, instead of availing the subject attribute of a subject's address, the obfuscated attribute may be a zip code or a state in which the subject lives. Whether an incomplete subject or an obfuscated subset is availed, cloud server 135 anonymizes a subject associated with the subject record.

IV.E. Chatbot Integration with Self-Learning Knowledge Base

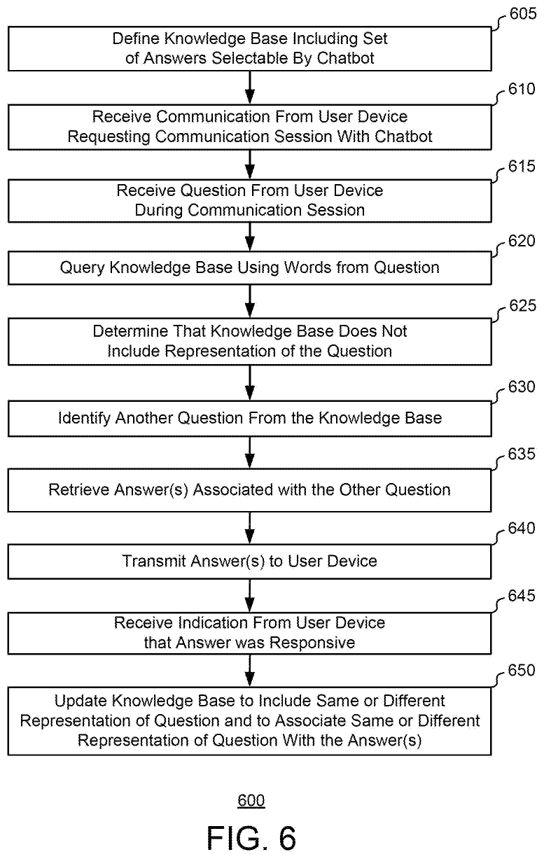

FIG. 6 is a flowchart illustrating process 600 for communicating with users using bot scripts, such as a chatbot. Process 600 may be performed by cloud server 135 for automatically linking new questions provided by users to existing questions in a knowledge base to provide a response to the new question. A chatbot may be configured to provide answers to questions associated with a condition.

At block 605, cloud server 135 defines a knowledge base, which includes a set of answers. The knowledge base may be a data structure stored in memory. The data structure stores text representing the set of answers to defined questions. Each answer may be selectable by a chatbot in response to a question received from a user device during a communication session. The knowledge base may be automatically defined (e.g., by retrieving text from a data source and parsing through the text using natural language processing techniques) or user defined (e.g., by a researcher or physician).

At block 610, cloud server 135 receives a communication from a particular user device. The communication corresponds to a request to initiate a communication session with a particular chatbot. For example, a physician or subject may operate a user device to communicate with a chatbot in a chat session. Cloud server 135 (or a module stored within cloud server 135) may manage or establish communication sessions between user devices and chatbots. At block 615, cloud server 135 receives a particular question from the particular user device during the communication session. The question can be a string of text that is processed using natural language processing techniques.

At block 620, cloud server 135 queries the knowledge base using at least some words extracted from the particular question. The words may be extracted from the string of text representing the particular question using natural language processing techniques. At block 625, cloud server 135 determines that the knowledge base does not include a representation of the particular question. In this case, the question received may be newly posed to a chatbot. At block 630, cloud server 135 identifies another question representation from the knowledge base. Cloud server 135 may identify another question representation by comparing the question received from the user device to the other question representations stored in the knowledge base. If a similarity is determined, for example, based on an analysis of the question representations using natural language processing techniques, then cloud server 135 identifies the other question representation.

At block 635, cloud server 135 retrieves an answer of the set of answers associated, in the knowledge base, with the other question representation. At block 640, the answer retrieved at block 635 is transmitted to the particular user device as an answer to the question received, even though the knowledge based did not include a representation of the question received. At block 645, cloud server 135 receives an indication from the particular user device. For example, the indication may be received in response to the user device indicating that the answer provided by the chatbot was responsive to the particular question.

At block 650, cloud server 135 updates the knowledge base to include the representation of the particular question or different representation of the particular question. For example, storing a representation of a question includes storing keywords included in the question in a data structure. Cloud server 135 may also associate the same or different representation of the particular question with the more answer transmitted to the particular user device.

In some embodiments, cloud server 135 accesses a subject record associated with the particular user device. Cloud server 135 determines a plurality of answers to the particular question. Cloud server 135 then selects an answer from the set of answers. The selection of the answer, however, is based at least in part on one or more values included in the subject record associated with the particular user device. For example, a value included in the subject record may represent a symptom recently experienced by the subject. The chatbot may be configured to select an answer that is dependent on the symptom recently experienced by the subject. In some instances, cloud server 135 may access a learn-to-rank machine-learning model that has been trained to predict an order for each answer in a set of answers. The learn-to-rank machine-learning model may be trained using a training set of answers. Each answer of the training set of answers may be labeled with one or more symptoms and a relevance score for that symptom. The relevance score may represent a relevance of the associated answer to a given symptom of the one or more symptoms. The relevance score may be user defined or automatically determined based on certain factors, such as frequency of a word (e.g., the word(s) for the symptom) in a training answer. The training set of answers may be different from the set of answers used when the chatbot is operational in a production environment. The learn-to-rank machine-learning model may learn how to order the set of answers (used in the production environment) in terms of relevance to a symptom (which is detected from the subject profile) based on the patterns learned by the learn-to-rank model (e.g., the patterns between the labeled training set of answers and the associated relevance scores for each symptom of one or more symptoms). The chatbot may select an answer from the set of answers used in the production environment based on the predicted ordering of the set of answers. In some instances, each answer of the set of answers may be associated with a tag or code indicating one or more symptoms that are associated with the answer. Cloud server 135 may compare the value that represents the symptom recently experienced by the subject with the tag or code associated with each answer.

V. A Network Environment Configured to Facilitate Intelligent Treatment Selection for Treating Subjects Diagnosed with SMA

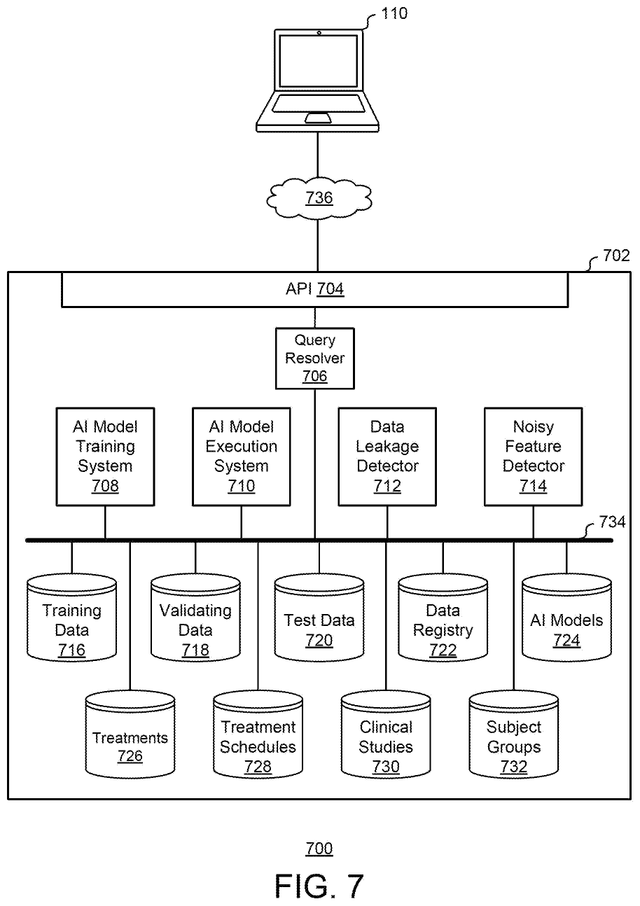

FIG. 7 is a block diagram illustrating an example of a network environment for deploying trained artificial-intelligence models to facilitate the subject-specific identification of treatments and treatment schedules for treating subjects with SMA, according to some aspects of the present disclosure. Network environment 700 can include user device 110 and AI system 702. User device 110 can interact with AI system 702 using network 736 (e.g., any public or private network), which facilitates the exchange of communications between user device 110 and AI system 702. AI system 702 may be another implementation of AI system 145, which is described with respect to FIG. 1. User device 110 can be operated by a user, such as a physician or other medical professional who is treating a subject diagnosed with SMA. User device 110 can transmit requests to AI system 702 using Application Programming Interface (API) 704 for triggering certain functionality (e.g., cloud-based services). While FIG. 7 illustrates a single user device 110, it will be appreciated that any number of user devices or other computing devices, such as cloud-based servers, may interact with AI system 702.

AI system 702 can be configured to perform certain predictive functionality, such as, for example, predicting suitable candidates for clinical studies, predicting a disease progression for a particular subject with SMA, or predicting a contextual treatment schedule specific to the particular subject. AI system 702 can perform the predictive functionality using, for example, AI model execution system 710. A number of data structures (e.g., databases) for storing data can facilitate the predictive functionality that AI system 702 can perform. In some implementations, the data structures can store training data 716, validating data 718, test data 720, subject records from data registry 722, AI models 724, treatments 726, treatment schedules 728, clinical studies 730, and subject groups identifiers 732. The various components of AI system 702 can communicate with each other using a communication network 734.

AI model training system 708 can facilitate the training of AI models using training data 716. For example, AI model training system 708 can execute code (e.g., executed by a processor, such as a physical or virtual CPU of a cloud-based server), which causes training data 716 to be inputted into learning algorithms. Learning algorithms can be executed to detect patterns or correlations between data points included in training data 716. The detected patterns or correlations can be stored as an AI model, which is trained to generate an output predictive of an outcome based on the stored patterns or correlations in response to receiving an input (e.g., of new, previously unseen input data, such as a subject record for a subject not included in the training data 716).

In some implementations as described in greater detail with respect to FIGS. 8 and 11, the output of a trained AI model can be predictive of a disease progression for a particular subject diagnosed with SMA. In other implementations, as described in greater detail with respect to FIGS. 9 and 12, the output of a trained AI model can be predictive of new or previously uninvestigated targets to investigate using new clinical studies and suitable candidate subjects for the new clinical studies. In other implementations as described in greater detail with respect to FIGS. 10 and 13, the output of a trained AI model can be predictive of a treatment selection for a particular subject with SMA.

The learning algorithms executed by AI system 702 may include any supervised, unsupervised, semi-supervised, reinforcement, and/or ensemble learning algorithms. Non-limiting examples of learning algorithms that can be executed by AI system 702 are included in Table 1 below. The selection of a learning algorithm by AI system 702 for training an AI model can be based on, for example, the type and size of at least a portion of training data 716 and the target predictive outcomes intended for the predictive functionality that AI system 702 can perform. The learning algorithms provided in Table 1 can be used for any of the methods described herein.

TABLE 1

| Model Type | Learning Algorithm |
|---|---|
| Text Analysis | N-Gram Extraction |
| | Word-to-Vector |
| | Preprocessing Text |
| | Feature Hashing |
| Regression Analysis | Neural Network Regression |
| | Decision Tree Regression |
| | Boosted Decision Tree |
| | Fast Forest Quantile |
| | Poisson Regression |
| | Linear Regression |
| | Bayesian Linear Regression |
| Image Classification | Convolutional Neural Network |
| | Generative Adversarial Network |
| | DenseNet |
| Clustering | K-Means |
| | Mean-Shift |
| | Density-Based Spatial Clustering of |
| | Applications with Noise |
| | Expectation-Maximization (EM) Clustering |
| | using Gaussian Mixture Models (GMM) |
| | Agglomerative Hierarchical Clustering |
| Multiclass Classification | Multiclass Logistic Regression |
| | Multiclass Boosted Decision Tree |
| | Multiclass Decision Forest |
| | Multiclass Neural Network |
| | One-vs-All Multiclass |
| Recommendation Models | Two-Class Support Vector Machine |
| | Two-Class Averaged Perceptron |
| | Two-Class Decision Forest |
| | Two-Class Logistic Regression |
| Anomaly Detection | PCA-Based Anomaly Detection |
| | Support Vector Machine |

In addition, during the process of training the various AI models, AI model training system 708 can interact with training data 716, validating data 718, and test data 720. Training data 716 is the data set that is inputted into the learning algorithm. The learning algorithm detects patterns, correlations, or relationships between data points within training data 716. However, the patterns, correlations, or relationships (e.g., the parameters) detected by the learning algorithm can overfit training data 716. Overfitting occurs when the analysis executed by the learning algorithm (e.g., which generated the patterns, correlations, or relationships) corresponds exactly or substantially exactly to training data 716. In this case, the analysis executed by the learning algorithms may not accurately serve as the basis of predicting new, previously unseen input data. Therefore, validating data 718 is a different data set from training data 716, and is used to modify the patterns, correlations, or relationships to prevent overfitting the training data 716. In cases where multiple learning algorithms are executed on training data 716, validating data 718 can be used to identify the learning algorithm with the highest performance on new input data (e.g., input data that is not included in training data 716). Validating data 718 can be used to generate an error function that can be evaluated to determine the performance of each learning algorithm on new input data. For example, the patterns, correlations, or relationships detected within training data 716 by each of the various learning algorithms can be stored in various AI models. The error function of each AI model on new input data can be evaluated using validating data 718. The AI model with the lowest error function can be selected. Lastly, test data 720 is another data set, which is independent from each of training data 716 and validating data 718. Test data 720 can be inputted into the selected AI model to test the overall performance of the selected AI model.

In some implementations, training data 716, validating data 718, and test data 720 can be segments across a single larger data set. For example, a data set can be segmented into three data subsets. The training data 716 can be one of the three data subsets, validating data 718 can be another one of the three data subsets, and test data 720 can be the last of the three data subsets. In some implementations, the data set that is segmented into three or more subsets can include any data or data type. Non-limiting examples of data or data types that can be included in the data set from which training data 716, validating data 718, and/or test data 720 are generated include radiological image data, MRI data, genomic profile data, clinical data (e.g., measurements, treatments, treatment responses, diagnoses, severity, medical history), subject-generated data (e.g., notes inputted by a subject with SMA), physician- or medical professional-generated data (e.g., physician notes), audio data representing phone recordings between a patient and a physician or other medical professional, administrative data, claims data, health surveys (e.g., Health Risk Assessment (HRS) Survey), third-party or vendor information (e.g., out of network lab results), public databases relevant to the subject (e.g., medical journals relevant to a subject's condition), subject demographics, immunizations, radiology reports, pathology reports, utilization information, metadata representing biological samples, social data (e.g., education level, employment status), community specifications, and so on. In some instances, at least some of the subject record can initially be identified via a communication (e.g., received at a care-provider device and/or remote server) from a device operated by the subject. In some implementations, at least some features of the subject record include or are based on one or more photographs (e.g., collected at a device of the subject). In some instances, at least some of the subject-specific data was initially identified via and/or was received from an electronic medical record corresponding to the subject.

AI model execution system 710 can be implemented using executable code that, when executed by a processor (e.g., a physical or virtual CPU of a cloud-based network, such as cloud network 130), executes an instance of a specific trained AI model to generate an output. The output can be predictive of certain outcomes relating to SMA because the AI model.

To illustrate and only as a non-limiting example, AI model execution system 710 receives a request from query resolver 706 (which originated from user device 110, operated by a user, such as a physician). The request is for predicting a disease progression for a particular subject with SMA. The request includes at least a portion of the subject record characterizing the particular subject (or an identifier of the subject record to enable retrieval of the subject record by another component). AI model execution system 710 evaluates the request and selects a trained Word-to-Vector model (stored in AI models data store 724) that is configured to generate predictions of disease progressions of subjects. AI model execution system 710 retrieves or accesses the Word-to-Vector model from AI models data store 724 and then passes input data (e.g., a numerical representation of the current state of the particular subject) into the retrieved AI model. AI model execution system 710 generates an output (e.g., a value or values, such as in an array) that can be used to determine the disease progression of the particular subject. The predictive functionality described in this example is further described with respect to FIGS. 8 and 11.

As another illustration and only as a non-limiting example, user device 110 transmits a request to AI system 702 to generate predictions of which group of subjects would be suitable candidates for enrollment in a new clinical study. AI system 702 retrieves or accesses a trained feature selection model and an auto grouping model. AI system 702 then inputs a set of numerical representations of subject records into the feature selection model and subsequently into the auto grouping model to generate a prediction of a group of subjects that would be suitable candidates for a new clinical study (e.g., a new clinical study stored in clinical studies data store 730). An identifier of the group of subjects predicted to be suitable candidates for enrollment in the new clinical study may be stored in subject groups data store 732. In some examples, AI system 702 can automatically identify groups of subjects who would be suitable candidates for a clinical study without needing to receive a request from user device 110. In other examples, AI system 702 can automatically identify a group of subjects based on a common feature of a group of subject records, and propose a new clinical study associated with the common feature, if one does not already exist. The predictive functionality described in this example is further described with respect to FIGS. 9 and 12.

As yet another illustration and only as a non-limiting example, user device 110 transmits a request to AI system 702 to predict a treatment selection and treatment schedule for a particular subject. AI system 702 retrieves or accesses a trained reinforcement model configured to select an optimal treatment workflow, including a multi-stage treatment and a schedule for the multi-stage treatment. AI system 702 inputs a vector representing characteristics of the particular subject into the trained reinforcement model to generate an output representing a specific multi-stage treatment (from amongst a plurality of single or multi-stage treatments stored in treatments data store 726 and treatment schedule data store 728) and a schedule for performing the multi-stage treatment. The predictive functionality described in this example is further described with respect to FIGS. 10 and 13.

Certain AI models can exhibit a technical problem of memorizing a portion of training data 716 during the training process. Memorizing a portion of training data 716 can occur when the trained AI model outputs a data element included in training data 716 as-is in response to receiving input data. Data leakage refers to an AI model outputting data elements as-is from the training data in response to an input of new, previously unseen data. In some cases, AI models memorize training data when the AI model is overfitted to the training data. An overfitted AI model memorizes noise contained in the training data (e.g., memorizing data elements from the training data that are not relevant to the task of learning). Thus, the AI model does not generalize predictions on new, previously unseen input data when the AI model exhibits data leakage.

Data leakage can violate privacy regulations if the training data includes sensitive or private data about subjects. To illustrate and as only a non-limiting example, training data 716 includes a subject record containing a value representing that the subject (who is characterized by the subject record) has a gene mutation linked with the early onset of Alzheimer's disease. The value representing the presence of the gene mutation for Alzheimer's disease is sensitive or private data. Therefore, various privacy laws and regulations prohibit the unauthorized disclosure of the subject's sensitive or private data (e.g., the Health Insurance Portability and Accountability Act (HIPAA)). If the trained AI model is overfitted to training data 716, however, a technical challenge arises in that the trained AI model is capable of leaking (e.g., unintentionally disclosing externally or to unauthorized users) the value representing that the subject has the gene mutation for Alzheimer's disease. In some scenarios, a privacy violation may occur if an adversary user device (e.g., operated by a user who is intentionally seeking to extract sensitive information from the AI model) can transmit inputs into the trained AI model and receive the corresponding outputs generated by the AI model. For example, if an adversary user device accesses the trained AI model using a public API, then the adversary user device can transmit inputs into the trained AI model and receive the outputs generated by the trained AI model. The adversary user device can then evaluate the various outputs received from the trained AI model to infer sensitive or private data about the training data used to train the AI model. Non-limiting examples of the sensitive or private data that can be inferred include the values indicating the presence of certain genetic mutations in a particular subject, the presence or absence of a subject record in the training data, the presence or absence of a particular subject in a particular clinical study, a correlation between the phenotypes presented by a particular subject and the genetic predisposition of the particular subject to developing a particular disease, such as SMA, characteristics of a particular subject's genetic profile, and any other sensitive or private data.

To solve the technical challenges with respect to data leakage described above, certain aspects and features of the present disclosure relate to configuring a data leakage detector 712 to detect and also to prevent data leakage when AI model execution system 710 executes any of the trained AI models stored in AI models data store 724. In some implementations, data leakage detector 712 can perform certain data leakage prevention protocols on training data 716, validating data 718, test data 720, and/or AI models 724. Performing data leakage prevention protocols on training data 716, validating data 718, test data 720, and/or AI models 724 can inhibit or prevent the leakage of sensitive data by trained AI models. Non-limiting examples of data leakage prevention protocols performed on data include encrypting sensitive or private data contained in subject records, data sanitization, data regularization, robust statistics, adversarial training, differential privacy, federated learning, homomorphic encryption, and other suitable techniques for inhibiting or preventing the leakage of sensitive data characterizing subjects.

Referring again to FIG. 7, a subject record can include data elements that characterize a subject feature using a large number of dimensions (e.g., hundreds or thousands of feature dimensions). Certain feature dimensions in a subject record may be useful for a target task, while other feature dimensions in the subject record may represent noisy data (e.g., features that are not useful for the target task). The high-dimensionality of subject records creates a technical challenge with respect to inputting the subject records (or the numerical representations thereof) as part of the predictive functionality provided by the various AI models associated with AI system 702. Certain aspects and features of the present disclosure relate to a noisy feature detector 714, which provides a solution to the technical challenges described above. In some implementations, noisy feature detector 714 can be configured to transform high-dimensionality subject records into reduced-dimensionality subject records by classifying a subset of subject features of the set of subject features contained in a subject record as noise. For example, the noisy feature detector 714 may execute a two-class classification model that is trained to classify subject features as either predictive for a target task or as noise. It will be appreciated that noisy feature detector 714 can also be a multi-class classification model that can classify subject features of a subject record into one or more of multiple classes (e.g., noise data, useful but not predictive for target task, and useful and predictive for target task). The reduction in dimensionality of subject records improves the computational efficiency of AI system 702 by reducing the number of feature dimensions of the subject records that AI model execution system 710 processes when providing the predictive functionality. Non-limiting examples of techniques for reducing the dimensionality of subject records include reducing features based on a criterion, reducing features based on feature category, feature selection techniques, eliminating features classified as noise by a trained classifier model, and other suitable techniques.

VI. A Network Environment Configured to Predict a Disease Progression for a Subject with SMA Using Artificial-Intelligence Techniques

FIG. 8 is a block diagram illustrating an example of a network environment for deploying a trained artificial-intelligence model to generate outputs predictive of disease progression for subjects diagnosed with SMA, according to some aspects of the present disclosure. Network environment 800 can include user device 110 and AI system 802. AI system 802 may be similar to AI system 702 illustrated in FIG. 7, however, the components of AI system 802 may differ from the components of AI system 702. In some implementations, AI system 802 can include API 808, query resolver 810, query text string 812, a trained word-to-vector model 814, progression prediction system 816, and communication network 818. The components of AI system 802 illustrated in FIG. 8 may be in addition to, in lieu of, or a part of any components of AI system 702 illustrated in FIG. 7. API 808 can be the same as API 704 illustrated in FIG. 7, and query resolver 810 can be the same as query resolver 706 illustrated in FIG. 7.

AI system 802 can be configured to generate an output that is predictive of the disease progression for a subject diagnosed with SMA. In some examples, AI system 802 generates the output automatically without needing to be prompted by a request from user device 110. In other examples, AI system 802 generates the output in response to receiving a request from user device 110. To illustrate, user device 110 (e.g., operated by a physician or other medical professional) can transmit a request to AI system 802. The request may be a request for AI system 802 to execute the predictive function configured to generate a prediction of the disease progression that a particular subject is likely to experience. In some examples, the request includes subject record 804 characterizing features of the particular subject. In other examples, the request includes an identifier of the particular subject, such that the identifier is used at a later time to retrieve subject record 804, which characterizes features of the particular subject. Regardless of how subject record 804 is accessed or retrieved, subject record 804 can include data elements representing a state of the particular subject. As a non-limiting example, the state of the particular subject may include text values, such as a diagnosis of the subject, the SMA type of the diagnosis, the phenotypes observed by a physician, any single-stage treatments performed on the particular subject, any multi-stage treatments performed on the subject, the amount of time that has elapsed between treatments of any kind, the genetic profile of the particular subject, clinical information characterizing the particular subject, and other suitable text values. Further, the state of the particular subject may represent a current state of the particular subject (e.g., the state of the particular subject at or near the time the request is transmitted by user device 110).

API 808 can be configured to enable user device 110 to interact with AI system 802. Accordingly, user device 110 can transmit the request (including the subject record 804) to AI system 802 using API 808. Query resolver 810 can receive the request from API 808, identify the trained AI model that can resolve the request, and then construct a query for the identified AI model. Query resolver 810 can identify that the request to predict the disease progression of the particular subject diagnosed with SMA can be resolved by transmitting an input into word-to-vector model 814 and providing the output to user device 110.

In some implementations, when query resolver 810 receives the request from user device 110, query resolver 810 can extract the subject record 804 from the request, if the request contains the subject record 804. In examples where the request includes a unique identifier identifying subject record 804, query resolver can extract the identifier of the subject record 804 and retrieve the subject record 804 from a data source, such as data registry 722 illustrated in FIG. 7. In some implementations, the subject record 804 may be anonymized to prevent AI system 802 from identifying the identity of the subject characterized by subject record 804. AI system query resolver 810 can then transmit the retrieved subject record 804 to query text string 812, which is configured to generate a partial word sequence using one or more features contained in the subject record 804.

To illustrate and only as a non-limiting example, subject record 804 includes at least four data elements. The first data element includes a first text value of “SMA positive,” representing a positive diagnosis for SMA. The second data element includes a second text value of “Type-III,” representing the type of SMA diagnosed. The third data element includes a third text value of “Proximal muscle weakness,” representing an observable phenotype of the particular subject. The fourth data element includes a fourth text value of “6 months,” representing an amount of time between first symptom onset experienced by the particular subject and a given time (e.g., time of receiving the request, 1^{st} of the current month). In some examples, each of the four data elements may include or be associated with a tag indicating an SMA-related data element, and only the four text values included in these four data elements may be processed by query text string 812. In other examples, the four data elements may be associated with a health state of the particular subject, and these four data elements may be processed by query text string 812. Query text string 812 can transform the four data elements into the partial word sequence, “[SMA positive],[Type-III],[Proximal muscle weakness],[6 months]”. The partial word sequence may be transmitted to query resolver 810 for passing onto word-to-vector model 814, or may be transmitted to word-to-vector model 814 directly.

Word-to-vector model 814 can be a machine-learning model trained to transform text-based word sequences into numerical representations for the purpose of enabling AI models to process the word sequences. The word-to-vector model can provide numerical representations for each word of a word sequence. The word embeddings of the words of the word sequence can be aggregated to numerically represent the word sequence. The numerical representations of multiple words in a word sequence can be compared to determine a relationship between the multiple words. Further, the aggregated numerical representations of words in a word sequence of two or more word sequences can be compared to determine the relationship between the two or more word sequences. Word-to-vector model 814 can be trained to learn the numerical representations of words in a word sequence using neural networks. Thus, the partial word sequence of “[SMA positive],[Type-III],[Proximal muscle weakness],[6 months]” is inputted into word-to-vector model 814. In some implementations, word-to-vector model 814 transforms the partial word sequence into a numerical representation (e.g., an N-dimensional words vector). The numerical representation of the partial word sequence can then be inputted into progression prediction system 816, which is trained to predict the remaining words in the partial word sequence. The remaining words that progression prediction system 816 generates as output representing the predicted sequence of progression of disease-related events, such as phenotypes or symptoms, are predicted to be experienced by the particular subject.

In some implementations, progression prediction system 816 can be a generative sequence model trained to perform certain language-related tasks, such as language-modeling and predictive sentence completion. A generative sequence model can model natural English language after being trained using all possible English word sequences. The generative sequence model can be trained to assign probabilities to words based on the sentences in which those words appeared. Using the assigned probabilities, generative sequence models can be configured to predict the remaining word or words that complete a partial word sequence (e.g., complete a partial sentence). To illustrate, a generative sequence model can be trained to predict that the word “hill” has a high probability of being the next word to complete the partial word sequence of “Jack and Jill went up the,” and that the word “there” has a low probability of being the next word to complete that partial word sequence because English grammar requires that the partial word sequence be followed by a noun.

In the context of predicting the disease progression of a particular subject diagnosed with SMA, progression prediction system 816 can execute a trained generative sequence model to generate predictions of the next words to complete a particular word sequence, where the predicted next words represent the predicted disease progression of the particular subject. Progression prediction system 816 can be trained using a training data set that includes a set of word sequences. Each word sequence in the set of word sequences represents a predicted disease-related event, such as a phenotype or a symptom, that a subject with SMA previously experienced. Table 2 below provides an illustrative example of a set of word sequences. Each word sequence in Table 2 is a sequence of single or multiple words (e.g., a single word, such as “[scoliosis]” or a grouping of multiple words, such as “[walks with cane]”) that represent the progression of disease events of subjects previously diagnosed with SMA.

TABLE 2

| | Word Sequence Representing SMA Disease |
|---|---|
| Anonymous Subject ID | Progression |
| ASDF2 | [walks with cane], [wheelchair bound] |
| FXQO5 | [loss of ambulation in childhood], [scoliosis], |
| | [difficulty swallowing], [respiratory infection] |
| ASPG4 | [weakness in muscles supporting femur], |
| | [wheel-chair bound], [back brace needed] |
| RIWEO | [loss of ambulation in adolescence], [wheelchair |
| | bound], [respiratory infection] |
| . . . | . . . |
| POMF7 | [loss of ambulation in childhood], [fingers |
| | tremble], [loss of tendon reflexes], [needs |
| | assistance to sit], [respiratory infection] |

The progression prediction system 816 can be trained, using the tracked disease progressions of subjects previously diagnosed with SMA (e.g., as shown in Table 2), to learn correlations between events along a longitudinal dimension of the disease progression of subjects. To illustrate, for example, the progression prediction system 816 can learn that subjects who experienced a loss of ambulation have a high probability (e.g., above a threshold probability) that the disease will progress to a respiratory infection, which can be triggered by a weakness of muscles supporting the spine. Accordingly, when the current state of a particular subject is a loss of ambulation, the progression prediction system 816 can predict that the disease progression of the particular subject is likely to include a respiratory infection by predicting the words that complete the partial word sequence of at least “loss of ambulation.” When the disease progression is defined as a word sequence, where each word or group of words in a word sequence represents a disease-related event in a progressive sequence of disease-related events, then the next disease-related events in the disease progression of a particular subject can be predicted by predicting the next words that complete a given partial word sequence.

Continuing with the above non-limiting example of the four data elements in subject record 804, progression prediction system 816 receives the input partial word sequence of “[SMA positive],[Type-III],[Proximal muscle weakness],[6 months]” (or the numerical representation of the partial word sequence). The progression prediction system 816 can generate an output partial word sequence that is predicted to complete the input partial word sequence. The output partial word sequence is a sequence of words that is predicted to complete the input partial word sequence of “[SMA positive],[Type-III], [Proximal muscle weakness],[6 months]” based on the historical disease progressions of previously treated subjects with SMA. The output partial word sequence in this non-limiting example is “[weakness in muscles supporting femur],[walks with cane],[difficulty sitting up from seated position],[wheelchair bound].” In other words, the output partial word sequence represents that the predicted disease progression of the particular subject includes: (1) weakness in muscles supporting the femur, then (2) need for cane to assist with walking, then (3) difficulty sitting up unassisted, and then (4) need for wheelchair for mobility during the remainder of life. Query resolver 810 can transmit the predicted disease progression 806, which is specific to the particular subject characterized by subject record 804, to user device 110 for further assessment by a user.

### VII. A Network Environment Configured to Automatically Define Subject Groups for Proposing New Clinical Studies Using Artificial-Intelligence Techniques

Clustering subject records of subjects with SMA involves identifying clusters of subject records that share a common subject feature. Clustering subject records can also identify groups of subjects who are similar to each other in some aspect, characteristic, or feature. The clustering of subject records, however, is technically challenging given the high-dimensionality of subject records. For example, a subject record can have hundreds of individual subject features (e.g., dimensions). Therefore, clustering highly-dimensional subject records is problematic with certain clustering techniques, such as k-means clustering. Certain aspects and features of the present disclosure provide a technical solution that enables the clustering of highly-dimensional subject records characterizing subjects with SMA, for example, for the purpose of defining groups of subjects who are suitable candidates for new or existing clinical studies.

FIG. 9 is a block diagram illustrating an example of a network environment for intelligently defining subject groups for new or existing clinical studies, according to some aspects of the present disclosure. Network environment 900 can include AI system 902 and data stores 904 through 908 for storing high-dimensionality subject records characterizing subjects. While FIG. 9 illustrates three data stores (e.g., data stores 904 through 908), it will be appreciated that FIG. 9 is exemplary, and thus, any number of data stores can be included in network environment 900. AI system 902 may be similar to AI system 702 illustrated in FIG. 7, however, the components of AI system 902 may differ from the components of AI system 702. The components of AI system 902 illustrated in FIG. 9 may be in addition to, in lieu of, or a part of any components of AI system 702 illustrated in FIG. 7. API 910 can be the same as API 704 illustrated in FIG. 7. Further, feature selection model 912 and subspace clustering system 914 may be stored in AI models data store 724 and may be executable by AI model execution system 710 illustrated in FIG. 7.

In some implementations, AI system 902 can be configured to automatically detect groups of subjects diagnosed with SMA who are candidates for existing clinical studies. In other implementations, AI system 902 can be configured to generate predictions of new treatment trails that did not previously exist and to identify subjects who would be target candidates for the new clinical studies. An existing or new clinical study, for example, may be a clinical trial that is designed to study the clinical outcomes of new treatments or diagnostic tests to determine the effectiveness of the new treatments or diagnostic tests. For example, an existing clinical study for SMA may be a clinical trial that studies the effect of low-dose celecoxib on SMN2 expression in subjects with SMA.

High-dimensionality subject records data stores 904 through 908 can store subject records across multiple entities. As a non-limiting example, subject records data store 904 is operated by a medical facility in the United States, subject records data store 906 is operated by a medical research facility in Italy, and subject records data store 908 is operated by a hospital in Canada. The subject records stored in subject records data store 904 characterize a first group of subjects having been treated at the medical facility in the United States. Further, the subject records stored in subject records data store 906 characterize a second group of subjects participating in clinical studies performed at the medical research facility in Italy. Lastly, the subject records stored in subject records data store 908 characterize a third group of subjects having been treated at the hospital in Canada. Regardless of whether the data stores 904 through 908 are geographically distributed across facilities or are co-located at a single facility, the subject records stored therein can be grouped using AI-based feature selection techniques for defining candidate subjects suitable for existing or new clinical studies.

Feature selection model 912 can be executable code representing an instance of any AI-based feature selection models, such as, for example, sparse logistic regression, least absolute shrinkage and selection operator (LASSO), univariate thresholding (e.g., l₀-norm minimization, l₁-norm minimization), least angle regression for LASSO, coordinate descent, proximal techniques, Elastic Net, fused or grouped LASSO, and other suitable feature selection techniques. Feature selection model 912 can be trained to identify which incomplete subset of subject features of the set of subject features of the subject records is relevant to a target task. As an illustrative example, the target task is identifying subjects who would be candidates for inclusion in a clinical study relating to Evrysdi™ (risdiplam, F. Hoffman-La Roche AG). The detection of suitability for the clinical study can be a trained characteristic of feature selection model 912. For instance, feature selection model 912 can be trained using a training data set of subject records that each include a label of either “Enroll” or “Do Not Enroll” for the clinical study. Based on the training of feature selection model 912, feature selection model 912 can learn which incomplete subset of the set of subject features is relevant for the clinical study. For example, feature selection model 912 can be trained to learn that subjects who were diagnosed with SMA Type-II and who are between the ages of 2 to 25 are suitable candidates for the clinical study, based on the patterns, correlations, and relationships detected in the training data set. Thus, feature selection model 912 can include the subject feature relating to “age” and the subject feature relating to “SMA Type” in the incomplete subset of the set of subject features. The incomplete subset of subject features may or may not be considered highly-dimensional. Once the relevant features are automatically extracted using feature selection model 912, the incomplete subset of subject features can be inputted into subspace clustering system 914.

Subspace clustering system 914 can be configured to execute subspace clustering techniques to identify clusters of subject records within different subspaces (e.g., a selection of one or more dimensions). Executing the subspace clustering techniques enables clusters of subject records to be formed. Clusters can be defined by a subset of subject features (e.g., a subject feature representing a dimensional aspect of a subject). To illustrate and only as a non-limiting example, the incomplete subset of subject features of subject records includes gene expression levels of 75 genes, including the SMN2 gene, after a treatment is performed on the subjects. Subspace clustering system 914 is trained to cluster subjects across the 75 genes (e.g., across 75 dimensions) of the incomplete subset of subject features. As part of clustering subjects across the 75 genes, subspace clustering system 914 forms three clusters of subjects relating to the expression of the SMN2 gene: “SMN2 expression above a threshold”, “SMN2 expression below a threshold”, and “No SMN2 expression.” For example, subspace clustering system 914 can identify a cluster of subjects who experienced an expression of the SMN2 gene at a level that exceeds a threshold, thereby indicating a potentially successful treatment. The identified cluster of subjects can then be associated with a group identifier, which is stored in subject group identifier system 916. Further, the identified cluster of subjects is determined to be suitable for an additional existing clinical study due to the expression of the SMN2 gene being at a level that exceeds the threshold. As another example, subspace clustering system 914 can identify a sub-cluster of the “No SMN2 expression” cluster. The sub-cluster includes subjects for which observable improvements in motor functioning were noted after a treatment was performed, and for which no SMN2 expression increase was detected after the treatment. If no existing clinical study exists for this sub-cluster of subjects, then subspace clustering system 914 can generate a proposal for a new clinical study be created to study the subjects who experienced improved motor functionality after the treatment and no increased expression in the SMN2 gene after the treatment.

VIII. The Cloud-Based Application Can Select an Optimal Treatment for a Subject with SMA, Given the Context of the Subject's Record

FIG. 10 is a block diagram illustrating an example of a network environment for deploying a trained reinforcement learner to select treatments, according to some aspects of the present disclosure. Network environment 1000 can include AI system 1002. AI system 1002 may be similar to AI system 702 illustrated in FIG. 7, however, the components of AI system 1002 may differ from the components of AI system 702. The components of AI system 1002 illustrated in FIG. 10 may be in addition to, in lieu of, or a part of any components of AI system 702 illustrated in FIG. 7. API 1008 can be the same as API 704 illustrated in FIG. 7, and query resolver 1010 can be the same as query resolver 706 illustrated in FIG. 7. Treatment selection system 1032 may be stored in AI models data store 724 and may be executable by AI model execution system 710 illustrated in FIG. 7.

In some implementations, AI system 1002 can be configured to select the optimal treatment for a particular subject from a group of treatments 1012 through 1030. Treatments 1012 through 1030 may represent the potential actions that a physician can undertake while treating a particular subject. To illustrate and only as a non-limiting example, treatment 1012 may be nusinersen (SPINRAZA), treatment 1014 may be providing a walking cane, treatment 1016 may be providing a wheelchair, treatment 1018 may be providing a dietary plan suitable for subjects with weakening jaw muscles, treatment 1020 may be Onasemnogene abeparvovec-xioi (Zolgensma), treatment 1022 may be a specialized mask or breathing apparatus to support weak respiratory muscles, treatment 1024 may be a feeding tube, treatment 1026 may be physical therapy, treatment 1028 may be a back brace, and treatment 1030 may be leg braces. Treatments may be multi-stage treatments, which can occur in a sequence over several phases or stages. While FIG. 10 illustrates treatments 1012 through 1030, it will be appreciated that any number of treatments may be performed as actions by or at the direction of a treating physician.

Treatment observations 1034 can be a data store storing historical observations across previously treatment subjects of outcomes in response to each of treatments 1012 through 1030. For example, a treatment observation of performing treatment 1012 on a subject may be that the SMN2 gene expression increased. As another example, a treatment observation of performing treatment 1014 may be that the support provided by a cane is insufficient to assist the subject in walking, given the progression of degeneration of the subject's thigh muscles (e.g., the rectus femoris muscle). In some examples, a survival probability associated with each treatment 1012 through 1030 may be stored in treatment observations 1034. For each treatment 1012 through 1030, the survival probability may be a value (e.g., a percentage) that represents a probability that the subject survives after undergoing the treatment. In other examples, the survival probability may also include a value representing a subject's quality of life after undergoing the treatment. In some implementations, the survival probability is automatically determined and updated as new treatment observations are stored in treatment observations data store 1034. For example, the survival probability is the number of subjects who survive a treatment, such as a surgery, after 30 days from the surgery. In some implementations, the survival probability may be inputted by a physician or subject after an assessment of the subjects health. In other examples, treatment observations data store 1034 can also store any side effects associated with each treatment 1012 through 1030.

Treatment selection system 1032 can be trained to learn the patterns, correlations, or relationships between each treatment 1012 through 1030 and the treatment observations stored in data store 1034. The treatment observations associated with each treatment 1012 through 1030 can represent a reward function associated with the treatment. The reward function can generate a “reward value,” such as a score of “5” out of “5,” for example, indicating that the treatment has a strong positive response in the subject. The “reward value” can also be a negative value, such as “−3” out of “5,” indicating that the treatment had a strong negative response in the subject. In some implementations, the reward value can be the increase in the expression of SMN2 in response to undergoing a gene therapy. The reward function can be designed to balance any short-term treatment observation with a long-term treatment observation. The short-term treatment observation and the long-term treatment observation can be transformed into a numerical value or vector (e.g., using a word-to-vector model). The short-term and long-term treatment observations can individually be weighted to reflect the balance between short-term observable outcomes and long-term observable outcomes. Treatment selection system 1032 can be trained to select a treatment from amongst treatments 1012 through 1030, such that the treatment is selected to maximize the reward function. Treatment selection system 1032 may be any reinforcement learning model, such as, for example, model-free reinforcement learning, policy optimization, policy gradient, model-based reinforcement learning, Q-function, Q-Table, importance sampling, U-curve, deep reinforcement, supervised reinforcement learning with a recurrent neural network, and other suitable reinforcement learning techniques.

To illustrate and only as a non-limiting example, a state of a subject may be characterized by subject record 1004, and an observable phenotype 1006 can be a phenotype of SMA observed in a subject having been diagnosed with SMA. The subject record 1004 and the phenotype 1006 can represent the current health state of a particular subject. The subject record 1004 and the phenotype 1006 are inputted into AI system 1002. API 1008 can be configured to enable the exchange of certain data between the AI system 1002 and external systems. Query resolver 1010 can transmit the subject record 1004 or phenotype 1006 of the particular subject to treatment selection system 1032 for selection of an optimal action. Treatment selection system 1032 can be executed to select a treatment from treatments 1012 through 1030 based on the reward function. Once a treatment is selected, such as treatment 1018, then AI system 1002 can transmit the selected treatment 1018 to a computing device for further assessment.

IX. The Cloud-Based Application Can Predict a Disease Progression for a Subject with SMA Using Artificial-Intelligence Techniques

FIG. 11 is a flowchart illustrating an example of a process for predicting the disease progression of subjects diagnosed with SMA, according to some aspects of the present disclosure. Process 1100 can be performed by any components illustrated in FIGS. 1, and 7-10. For example, process 1100 can be performed by AI system 802. Further, process 1100 can be performed to execute an AI model that generates output predictive of the progression of phenotypes, symptoms, or other disease-related events for a particular subject diagnosed with SMA.

Process 1100 begins at block 1105, where AI system 802, for example, accesses or retrieves a subject record corresponding to a particular subject (e.g., a subject being treated at a hospital). The subject record (e.g., an electronic medical record or an electronic health record) can include any number of features (e.g., data elements containing values, such as immunizations, history of medication, age, demographics) collected from or on behalf of the subject. The subject record can include a set of features that characterize aspects of the subject. For example, the subject record can include, among a multitude of other features, a feature indicating that the subject has been diagnosed with SMA Type-III.

Non-limiting examples of features that can be contained in a subject record include radiological image data, MRI data, genomic profile data, clinical data (e.g., measurements, treatments, treatment responses, diagnoses, severity, medical history), subject-generated data (e.g., notes inputted by a subject with SMA), physician- or medical professional-generated data (e.g., physician notes), audio data representing phone recordings between a patient and a physician or other medical professional, administrative data, claims data, health surveys (e.g., Health Risk Assessment (HRS) Survey), third-party or vendor information (e.g., out of network lab results), public databases relevant to the subject (e.g., medical journals relevant to a subject's condition), subject demographics, immunizations, radiology reports, pathology reports, utilization information, metadata representing biological samples, social data (e.g., education level, employment status), community specifications, and so on.

At block 1110, AI system 802 can extract features that are related to SMA or to the subject's diagnosis of SMA. In some implementations, any feature that relates to the diagnosis or treatment of a subject with SMA can be tagged as being relevant for SMA. For example, features that relate to the results of motor function tests, such as the 6-Minute Walking Test or the Wolf Motor Function Test, can be tagged as being relevant for SMA diagnostics or treatments. Tagging a feature in a subject record can include storing a code (e.g., “0000” or “SMA-TAG”) within a data element, such that the code is detectable and readable by AI system 802. The code can be interpreted by AI system 802 as a feature that relates to an SMA characteristic. A user (e.g., a physician) may tag features individually or the features can be tagged automatically upon entry of data into the features.

In some implementations, the features may not be tagged as being relevant to SMA diagnostics or treatments, however, instead, AI system 802 can automatically classify which features relate to SMA diagnostics or treatments. For example, AI system 802 can store a classification model that is trained to recognize features that relate to SMA diagnostics or treatments (or any other relation to SMA). Any classifier model can be used, including, for example, logistic regression, Naive Bayes, Stochastic Gradient Descent, K-Nearest Neighbors, decision tree models, random forest models, support vector machines (SVM), and any other suitable model of model.

At block 1115, AI system 802 can generate a partial word sequence using the SMA-related features identified at block 1110. To illustrate, the features that are identified as corresponding to SMA (at block 1110) include the following: [“SMA Type-II”],[“4 months since symptom onset”],[“loss of ambulation at age 2”],[“current age 3”],[“difficulty sitting upright”]. AI system 802 can execute query text string 812 to transform the features of the subject record into a partial word sequence, such as [SMA Type-II, 4 months since symptom onset, loss of ambulation at age 2, current age 3, difficulty sitting upright]. The partial word sequence is a sentence comprising the SMA-related features identified at block 1110 separated by commas.

The partial word sequence is partial because it represents a current health state of the subject with respect to the subject's SMA diagnosis. At block 1120, the AI system 802 receives the partial word sequence as input and transforms the partial word sequence into a vector representation using a word-to-vector model (e.g., Word2Vec).

Once the partial word sequence is transformed into a vector representation, certain predictive functionality can be performed using the partial word sequence. At block 1125, in the context of predicting the disease progression (e.g., the progression of SMA phenotypes) for a particular subject diagnosed with SMA, AI system 802 can input the vector representation of the partial word sequence into a trained generative sequence model (e.g., an natural language processing (NLP) model). At block 1130, the generative sequence model can generate a prediction of the one or more next words (e.g., a completion word or phrase) that is predicted to follow the partial word sequence (e.g., to complete the partial word sequence). The predicted next words represent the subject's predicted disease progression of SMA phenotypes, symptoms, diagnostics, or treatments over a period of time. The prediction of the next words that are likely to complete the partial word sequence represents the next SMA phenotypes that the subject is predicted to exhibit. For example, each next word outputted by the generative sequence model represents a predicted phenotype, symptom, treatment, and/or disease-related event that the subject is predicted to experience or exhibit. The prediction of the next words are based on a training data set that includes word sequences representing the progression of disease-related events, such as the predicted change in phenotypes or symptoms, of previously-treated subjects with SMA.

At block 1135, matching techniques, such as word matching, can be performed to fit the predicted completed word sequence with existing disease progression to identify previously treated subjects who experienced the same or similar disease progression. Additionally, fitting the predicted completed word sequence to an existing disease progression of another subject can also be performed to identify a physician treating the other subject who exhibited the same or similar disease progression. At block 1140, if the predicted disease progression satisfies an early treatment condition, then process 1100 can proceed to block 1145. However, if the predicted disease progression does not satisfy the early treatment condition, then process 1100 proceeds to block 1155. In some implementations, the early treatment condition may be a rule that is used to evaluate whether the predicted progression of the SMA phenotypes indicates a health risk over a future time period, such as over the next 6 months. For example, if the predicted progression of SMA phenotypes for a subject diagnosed with SMA is loss of ambulation in the next 4 months, then AI system 802 may interpret the predicted progression as satisfying the early treatment condition. In this case, at block 1145, AI system 802 queries a data store, such as data registry 722 for an identifier of physicians (e.g., who are employed by the same hospital or who have given their consent for being searchable for this purpose) who previously treated subjects with the same or similar disease progression.

At block 1150, AI system 802 can automatically generate and transmit a communication (e.g., an email) to a user device associated with the identified physician. The communication can be a request for a communication session to be initiated between the physician treating the subject and the physician who previously treated other subjects with similar disease progression (identified at block 1145). For example, during the communication session, the physicians can discuss the treatments that were performed on the other subjects and the clinical outcomes. The information provided by the physician identified at block 1145 can assist the treating physician with a treatment schedule for the subject before the symptoms occur according to the predicted progression of SMA phenotypes. When the early treatment condition is not satisfied (e.g., when the predicted progression of SMA phenotypes is mild or is not predicted to occur for years), then (at block 1155) AI system 802 can retrieve the subjects records corresponding to the subjects who share a similar or the same predicted disease progression and (at block 1160) display the associated treatments and treatment schedules on a user device.

### X. The Cloud-Based Application Can Automatically Define Subject Groups for Proposing New Clinical Studies Using Artificial-Intelligence Techniques

FIG. 12 is a flowchart illustrating an example of a process for intelligently defining subject groups for new or existing clinical studies, according to some aspects of the present disclosure. Process 1200 can be performed by any components illustrated in FIGS. 1, and 7-10. For example, process 1200 can be performed by AI system 902. Further, process 1200 can be performed to execute AI models that automatically generate reduced-dimensionality subject records and perform subspace clustering on the subject records to identify candidate subjects for new or existing clinical studies.

Process 1200 begins at block 1210, where AI system 902 accesses subject records stored in the data registry, for example, data registry 722. The subject records can be accessed automatically on a regular or irregular time interval or in response to a user input triggering the predictive functionality described in greater detail with respect to FIG. 12. At block 1220, some (e.g., not all) or all the subject records stored in the data registry can be transformed into numerical representations (e.g., vector representations) using various implementations described herein (e.g., described with respect to FIGS. 1-6). The subject records may be transformed or vectorized into numerical representations in advance or in real-time or substantially real-time with the performance of block 1210.

At block 1230, AI system 902 can perform AI-based feature selection on the subject records to select a subset of salient features from the numerical representations of the subject records. For example, given the high-dimensionality of subject records (e.g., with potentially hundreds of features), a feature selection model can be trained to detect and select features in subject records that are important for performing a target task, such as identifying candidate subjects for new or existing clinical studies. At block 1240, for each subject record accessed at block 1210, AI system 902 can generate a reduced-dimensionality numerical representation of the automatically selected salient features of the subject record.

The feature selection performed at block 1230 can be performed using any AI-based feature selection models, such as, for example, sparse logistic regression, least absolute shrinkage and selection operator (LASSO), univariate thresholding (e.g., l₀-norm minimization, l₁-norm minimization), least angle regression for LASSO, coordinate descent, proximal techniques, Elastic Net, fused or grouped LASSO, and other suitable feature selection techniques. The AI-based feature selection model can be trained to identify which incomplete subset of subject features of the set of subject features of the subject records is relevant to or for performing a target task.

To illustrate and only as a non-limiting example, the target task is identifying subjects who would be suitable candidates for inclusion in a clinical study relating to Evrysdi™ (risdiplam, F. Hoffman-La Roche AG). Detecting whether or not a subject is a suitable candidate for the clinical study can be a trained capability of the feature selection model. The feature selection model can be trained using a training data set of subject records that each include a label of either “suitable” or “not suitable” for the existing clinical study. Based on the patterns, correlations, and relationships learning during the training process, the feature selection model can learn which incomplete subset of the set of subject features is relevant for the clinical study. For example, the feature selection model can be trained to learn that subjects who were diagnosed with SMA Type-II and who are between the ages of 2 to 25 are suitable candidates for the clinical study, based on the patterns, correlations, and relationships detected in the training data set. Thus, the feature selection model can include the subject feature relating to “age” and the subject feature relating to “SMA Type” in the incomplete subset of the set of subject features.

At block 1250, AI system 902 can execute a protocol for automatically defining subject groups for new or existing clinical studies. In some implementations, the subject groups can be defined based on clustering of the reduced-dimensionality subject records (or the numerical representations thereof). The reduced-dimensionality subject records can still be challenging to process using techniques, such as k-means clustering. Accordingly, for example, the reduced-dimensionality subject records can be clustered in subspaces according to the various remaining dimensions of features. Subspace clustering be executed to identify clusters of subject records within different subspaces (e.g., a selection of one or more dimensions). Executing the subspace clustering techniques enables clusters of subject records to be formed. Clusters can be defined by a subset of subject features (e.g., a subject feature representing a dimensional aspect of a subject).

At block 1260, AI system 902 can generate a clinical study effectivity parameter to represent the effectiveness of a new or existing clinical study on the automatically defined subject groups. In some implementations, the clinical-study effectivity parameter may be a numerical value representing a degree to which the features of a subject group (defined at block 1250) is relevant to the features of a particular existing clinical study. A trained classification model can be used to classify the features associated with a subject as “effective” or “not effective” because on the clinical outcomes included in the clinical studies. The outputted classification can also be associated with a confidence or relevance parameter, which is also outputted by the classification model. If no existing clinical study exists for a subject group, and if the subject group is classified as having features that are likely to be “effective” for a clinical study, then AI system 902 can generate a proposal for a new clinical study to be created to study the subjects in the subject group. At block 1270, a subject group is selected for a new or existing treatment file based on the clinical study effectivity parameter generated at block 1260.

XI. The Cloud-Based Application Can Select an Optimal Treatment for a Subject with SMA, Given the Context of the Subject's Record

FIG. 13 is a flowchart illustrating an example of a process for deploying artificial-intelligence models to facilitate the selection of treatments to perform on subjects diagnosed with SMA, according to some aspects of the present disclosure. Process 1300 can be performed by any components illustrated in FIGS. 1 and 7-10. For example, process 1300 can be performed by AI system 1002. Further, process 1300 can be performed to execute reinforcement learning models trained to automatically select treatments to maximize a reward function, such as an amount of improvement in SMN2 expression.

Process 1300 beings at block 1310, where AI system 1002 accesses or retrieves a subject record stored in the data registry, for example, data registry 722. The subject record may characterize a particular subject who has been diagnosed with SMA. At block 1220, the subject record accessed or retrieved at block 1210 can be transformed into numerical representations (e.g., vector representations) using various implementations described herein (e.g., described with respect to FIGS. 1-6). The subject records may be transformed or vectorized into numerical representations in advance or in real-time or substantially real-time with the performance of block 1210.

At block 1330, AI system 1002 can generate a context vector that represents a context of the state of the particular subject's health. For example, a context vector is a fixed length vector that can contextualize the state of the subject record of the particular subject in numerical form. At block 1340, the context vector representing the particular subject can be inputted into a treatment selection system, which includes a reinforcement learner that learns to reinforce selected action (e.g., treatments) when a reward is received in response to performing the selected action. The treatment selection system may be any reinforcement learning model, such as, for example, model-free reinforcement learning, policy optimization, policy gradient, model-based reinforcement learning, Q-function, Q-Table, importance sampling, U-curve, deep reinforcement, supervised reinforcement learning with a recurrent neural network, and other suitable reinforcement learning techniques.

At block 1350, the treatment selection system can select an action, such as performing a gene therapy for increasing the expression of the SMN protein. The treatment selection system can intelligently select the treatment, from amongst a group of treatments, based on a prediction of the reward to be received. To illustrate, during the training process, the treatment selection system detects a pattern within the treatment observations indicating that subjects within the ages of 10 and 20 and treated with a first treatment (e.g., risdiplam) are likely to experience a 15%-20% increase in the expression of the SMN protein; subjects between the ages 2 and 10 and treated with a second treatment (e.g., nusinersen) are likely to experience a 3% increase in the expression of the SMN protein; and subjects between 5 and 12 and treated with a third treatment of weekly physical therapy are likely to experience an increase of 23% in their 6-Minute Walking Test score (indicating significant increase in motor functions). When a subject is 7 years old, the treatment selection system intelligently selects a treatment between the first treatment, the second treatment, and the third treatment based on the predicted reward. The treatment selection system selects the treatment to maximize the potential reward from the action. If the reward function is configured to maximize the percentage of increase in the expression of the SMN protein, then the treatment selection system selects the second treatment for the 7-year-old subject because this treatment offers the best reward with respect to the increase in SMN protein expression. However, if the reward function is configured to maximize the increase in motor functioning scores, for example, the 6-Minute Walking Test score, then the treatment selection may select the third treatment for the 7-year-old subject to maximize the reward.

Whichever treatment is selected at block 1360, the treatment selection system receives a response signal after the selected treatment is performed. For example, if the selected treatment is a dosage of nusinersen, then the response signal (whenever available after the treatment) would include the detected increase in the expression of the SMN protein in the subject. As another example, if the selected treatment is weekly physical therapy, the response signal (whenever available after the treatment) would include the percentage of improvement in the subject's 6-Minute Walking Test score. At block 1370, the treatment observations of the treatment selection system are updated with the response signal.

### XII. Additional Considerations

Some embodiments of the present disclosure include a system including one or more data processors. In some embodiments, the system includes a non-transitory, computer-readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory, machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

### XIII. Additional Examples

As used below, any reference to a series of examples is to be understood as a reference to each of those examples disjunctively (e.g., “Examples 1-4” is to be understood as “Examples 1, 2, 3, or 4”).

Example 1 is a computer-implemented method comprising: retrieving a subject record associated with a subject, the subject record including a set of features characterizing the subject, and the subject having been diagnosed with spinal muscular atrophy (SMA); extracting a subset of the set of features included in the subject record, each feature of the subset of the set of features being associated with an SMA characteristic; generating a partial word sequence by combining the subset of the set of features into a sequence of one or more words, each word of the one or more words representing a feature of the subset of features; transforming the partial word sequence into a numerical representation using a trained word-to-vector model; inputting the numerical representation of the partial word sequence into a natural language processing (NLP) model having been trained to predict a completion word or phrase for completing the partial word sequence; generating, based on the completion word or phrase outputted by the NLP model, a disease progression representing the predicted phenotype or symptoms for the subject over a future timeline (e.g., over the next year, the next 5 years, the next 10 years); and outputting an indication that the subject is predicted to exhibit the one or more SMA phenotypes included in the disease progression.

Example 2 is the computer-implemented method of example 1, further comprising: determining that the predicted progression of the one or more SMA phenotypes specific to the subject satisfies an early treatment condition, wherein satisfying the early treatment condition is indicative of a recommendation to perform a treatment before the subject exhibits an SMA phenotype of the one or more SMA phenotypes.

Example 3 is the computer-implemented method of examples 1-2, wherein when the predicted progression of the one or more SMA phenotypes satisfies the early treatment condition: identify an existing disease progression associated with an anonymized subject, the existing disease progression matching the predicted progression of the one or more SMA phenotypes specific to the subject, and the anonymized subject having been diagnosed with SMA; identify a user who training the anonymized subject associated with the existing disease progression; and transmit a communication to a user device associated with the user, the communication requesting treatment recommendations for the subject.

Example 4 is the computer-implemented method of examples 1-3, wherein when the predicted progression of the one or more SMA phenotypes does not satisfy the early treatment condition: identify an existing disease progression associated with an anonymized subject, the existing disease progression matching the predicted progression of the one or more SMA phenotypes specific to the subject, and the anonymized subject having been diagnosed with SMA; retrieving an anonymized subject record characterizing the anonymized subject; extracting a treatment schedule from the anonymized subject record; and transmitting the treatment schedule to a user device.

Example 5 is the computer-implemented method of examples 1-4, further comprising: matching the completion word or phrase associated with the subject to another one or more SMA phenotypes associated with another subject having been previously treated for SMA; retrieving an anonymized subject record characterizing the other subject; extracting a treatment schedule from the anonymized subject record; and transmitting the treatment schedule to a user device.

Example 6 is the computer-implemented method of examples 1-5, wherein the completion word or phrase is predicted as a next word in a complete word sequence including the partial word sequence, and wherein the completion word or phrase represents an SMA phenotype.

Example 7 is the computer-implemented method of examples 1-6, wherein the disease progression is output at a computing device of the subject using a chatbot.

Example 8 is the computer-implemented method of examples 1-7, the subject record includes data identified in an electronic medical record corresponding to the subject.

Example 9 is the computer-implemented method of examples 1-8, wherein the subject record corresponding to the subject includes a diagnosis of SMA Type-I, SMA Type-II, SMA Type III, or SMA Type-IV.

Example 10 is the computer-implemented method of examples 1-9, wherein training the NLP model further comprises: collecting a training data set including a set of subject records, each subject record of the set of subject records corresponding to another subject diagnosed with SMA, and each subject record of the set of subject record including one or more features representing a progression of SMA phenotypes during a time period; executing a learning algorithm associated with a generative sequence model using the training data set, wherein the learning algorithm detects patterns associated with the progression of SMA phenotypes exhibited by a set of subjects corresponding to the set of subject records; and generating the NLP model in response to executing the learning algorithm associated with the generative sequence model using the training data set.

Example 11 is the computer-implemented method of examples 1-10, further comprising: detecting data leakage associated with the NLP model, the data leakage exposing a feature of the set of features included in the subject record characterizing the subject; and in response to detecting data leakage associated with the NLP model, executing a data leakage prevention protocol that prevents or blocks exposure of the feature of the set of features included in the subject record.

Example 12 is the computer-implemented method of examples 1-11, wherein executing the data leakage prevention protocol includes re-training the NLP model according to a differential privacy model.

Example 13 is the computer-implemented method of examples 1-12, further comprising: generating, using a feature selection model, a reduced-dimensionality subject record characterizing the subject, the reduced-dimensionality subject record removing one or more features from the set of features included in the subject record, the one or more features being characterized as noise.

Example 14 is a system, comprising: one or more processors; and a non-transitory, computer-readable storage medium containing instructions which, when executed on the one or more processors, cause the one or more processors to perform part or all of one more of Examples 1-13 disclosed above.

Example 15 is a computer-program product tangibly embodied in a non-transitory, machine-readable storage medium, including instructions configured to cause one or more data processors to perform part or all of one or more of Examples 1-13 disclosed above.

1. A computer-implemented method comprising:
retrieving a subject record associated with a subject, the subject record including a set of features characterizing the subject, and the subject having been diagnosed with spinal muscular atrophy (SMA);
extracting a subset of the set of features included in the subject record, each feature of the subset of the set of features being associated with an SMA characteristic;
generating a partial word sequence by combining the subset of the set of features into a sequence of one or more words, each word of the one or more words representing a feature of the subset of features;
transforming the partial word sequence into a numerical representation using a trained word-to-vector model;
inputting the numerical representation of the partial word sequence into a natural language processing (NLP) model having been trained to predict a completion word or phrase for completing the partial word sequence;
generating, based on the completion word or phrase outputted by the NLP model, a disease progression representing a predicted progression of one or more SMA phenotypes specific to the subject over a period of time; and
outputting an indication that the subject is predicted to exhibit the one or more SMA phenotypes included in the disease progression.

2. The computer-implemented method of claim 1, further comprising:
determining that the predicted progression of the one or more SMA phenotypes specific to the subject satisfies an early treatment condition, wherein satisfying the early treatment condition is indicative of a recommendation to perform a treatment before the subject exhibits an SMA phenotype of the one or more SMA phenotypes.

3. The computer-implemented method of claim 1 further comprising, when the predicted progression of the one or more SMA phenotypes satisfies the early treatment condition:
identifying an existing disease progression associated with an anonymized subject, the existing disease progression matching the predicted progression of the one or more SMA phenotypes specific to the subject, and the anonymized subject having been diagnosed with SMA;
identifying a user who training the anonymized subject associated with the existing disease progression; and
transmitting a communication to a user device associated with the user, the communication requesting treatment recommendations for the subject.

4. The computer-implemented method of claim 1 further comprising, when the predicted progression of the one or more SMA phenotypes does not satisfy the early treatment condition:
identifying an existing disease progression associated with an anonymized subject, the existing disease progression matching the predicted progression of the one or more SMA phenotypes specific to the subject, and the anonymized subject having been diagnosed with SMA;
retrieving an anonymized subject record characterizing the anonymized subject;
extracting a treatment schedule from the anonymized subject record; and
transmitting the treatment schedule to a user device.

5. The computer-implemented method of claim 1, further comprising:
matching the completion word or phrase associated with the subject to another one or more SMA phenotypes associated with another subject having been previously treated for SMA;
retrieving an anonymized subject record characterizing the other subject;
extracting a treatment schedule from the anonymized subject record; and
transmitting the treatment schedule to a user device.

6. The computer-implemented method of claim 1, wherein the completion word or phrase is predicted as a next word in a complete word sequence including the partial word sequence, and wherein the completion word or phrase represents an SMA phenotype.

7. The computer-implemented method of claim 1, wherein the disease progression is output at a computing device of the subject using a chatbot.

8. The computer-implemented method of claim 1, wherein the subject record includes data identified in an electronic medical record corresponding to the subject.

9. The computer-implemented method of claim 1, wherein the subject record corresponding to the subject includes a diagnosis of SMA Type-I, SMA Type-II, SMA Type III, or SMA Type-IV.

10. The computer-implemented method of claim 1, wherein training the NLP model further comprises:
collecting a training data set including a set of subject records, each subject record of the set of subject records corresponding to another subject diagnosed with SMA, and each subject record of the set of subject record including one or more features representing a progression of SMA phenotypes during a time period;
executing a learning algorithm associated with a generative sequence model using the training data set, wherein the learning algorithm detects patterns associated with the progression of SMA phenotypes exhibited by a set of subjects corresponding to the set of subject records; and
generating the NLP model in response to executing the learning algorithm associated with the generative sequence model using the training data set.

11. The computer-implemented method of claim 1, further comprising:
detecting data leakage associated with the NLP model, the data leakage exposing a feature of the set of features included in the subject record characterizing the subject; and
in response to detecting data leakage associated with the NLP model, executing a data leakage prevention protocol that prevents or blocks exposure of the feature of the set of features included in the subject, record.

12. The computer-implemented method of claim 1, wherein executing the data leakage prevention protocol includes re-training the NLP model according to a differential privacy model.

13. The computer-implemented method of claim 1, further comprising:
generating, using a feature selection model, a reduced-dimensionality subject record characterizing the subject, the reduced-dimensionality subject record removing one or more features from the set of features included in the subject record, the one or more features being characterized as noise.

14. A system, comprising:
one or more processors; and
a non-transitory, computer-readable storage medium containing instructions which, when executed on the one or more processors, cause the one or more processors to perform a set of actions including:
retrieving a subject record associated with a subject, the subject record including a set of features characterizing the subject, and the subject having been diagnosed with spinal muscular atrophy (SMA);
extracting a subset of the set of features included in the subject record, each feature of the subset of the set of features being associated with an SMA characteristic;
generating a partial word sequence by combining the subset of the set of features into a sequence of one or more words, each word of the one or more words representing a feature of the subset of features;
transforming the partial word sequence into a numerical representation using a trained word-to-vector model;
inputting the numerical representation of the partial word sequence into a natural language processing (NLP) model having been trained to predict a completion word or phrase for completing the partial word sequence;
generating, based on the completion word or phrase outputted by the NLP model, a disease progression representing a predicted progression of one or more SMA phenotypes specific to the subject over a period of time; and
outputting an indication that the subject is predicted to exhibit the one or more SMA phenotypes included in the disease progression.

15. A computer-program product tangibly embodied in a non-transitory, machine-readable storage medium, including instructions configured to cause one or more data processors to perform a set of actions including:
retrieving a subject record associated with a subject, the subject record including a set of features characterizing the subject, and the subject having been diagnosed with spinal muscular atrophy (SMA);
extracting a subset of the set of features included in the subject record, each feature of the subset of the set of features being associated with an SMA characteristic;
generating a partial word sequence by combining the subset of the set of features into a sequence of one or more words, each word of the one or more words representing a feature of the subset of features;
transforming the partial word sequence into a numerical representation using a trained word-to-vector model;
inputting the numerical representation of the partial word sequence into a natural language processing (NLP) model having been trained to predict a completion word or phrase for completing the partial word sequence;
generating, based on the completion word or phrase outputted by the NLP model, a disease progression representing a predicted progression of one or more SMA phenotypes specific to the subject over a period of time; and
outputting an indication that the subject is predicted to exhibit the one or more SMA phenotypes included in the disease progression.

16. The system of claim 14, wherein the set of actions further includes:
determining that the predicted progression of the one or more SMA phenotypes specific to the subject satisfies an early treatment condition, wherein satisfying the early treatment condition is indicative of a recommendation to perform a treatment before the subject exhibits an SMA phenotype of the one or more SMA phenotypes.

17. The system of claim 14, wherein the set of actions further includes, when the predicted progression of the one or more SMA phenotypes satisfies the early treatment condition:
identifying an existing disease progression associated with an anonymized subject, the existing disease progression matching the predicted progression of the one or more SMA phenotypes specific to the subject, and the anonymized subject having been diagnosed with SMA;
identifying a user who training the anonymized subject associated with the existing disease progression; and
transmitting a communication to a user device associated with the user, the communication requesting treatment recommendations for the subject.

18. The system of claim 14, wherein the set of actions further includes, when the predicted progression of the one or more SMA phenotypes does not satisfy the early treatment condition:
identifying an existing disease progression associated with an anonymized subject, the existing disease progression matching the predicted progression of the one or more SMA phenotypes specific to the subject, and the anonymized subject having been diagnosed with SMA;
retrieving an anonymized subject record characterizing the anonymized subject;
extracting a treatment schedule from the anonymized subject record; and
transmitting the treatment schedule to a user device.

19. The system of claim 14, wherein the set of actions further includes:
matching the completion word or phrase associated with the subject to another one or more SMA phenotypes associated with another subject having been previously treated for SMA;
retrieving an anonymized subject record characterizing the other subject;
extracting a treatment schedule from the anonymized subject record; and
transmitting the treatment schedule to a user device.

20. The system of claim 14, wherein the completion word or phrase is predicted as a next word in a complete word sequence including the partial word sequence, and wherein the completion word or phrase represents an SMA phenotype.
